(19) United States
(12) Patent Application Publication
(10) Pub. No.: US 2022/0010380 A1
(43) Pub. Date: Jan. 13, 2022

(54) COMPOSITIONS AND METHODS RELATED TO DIFFERENTIALLY METHYLATED DNA SEQUENCES ASSOCIATED WITH MONOALLELIC GENE EXPRESSION AND DISEASE

(71) Applicant: North Carolina State University, Raleigh, NC (US)

(72) Inventors: Cathrine Hoyo, Raleigh, NC (US); David Skaar, Raleigh, NC (US); Dereje Jima, Raleigh, NC (US); Randy L. Jirtle, Raleigh, NC (US)

(73) Assignee: North Carolina State University, Raleigh, NC (US)

(21) Appl. No.: 17/372,113

(22) Filed: Jul. 9, 2021

Related U.S. Application Data
(60) Provisional application No. 63/050,086, filed on Jul. 9, 2020.

(51) Int. Cl. C12Q 1/6883

(52) U.S. Cl. CPC C12Q 1/6883; C12Q 2600/154

(57) ABSTRACT

Provided are compositions and methods for determining an imprinting status of a gene subject to parent-of-origin, monoalleleic expression in a subject, and for using the information derived therefrom to detect a presence of and/or a susceptibility to a medical condition associated with monoallelic expression in a subject, predict a susceptibility to future development of a medical condition prior to the onset of any symptoms, monitor the development of such a medical condition and/or the effectiveness of a treatment, and for treating such a medical condition. In some embodiments, a composition for use in the disclosed methods is nucleic acid array, which in some embodiments can include one or more interrogatable nucleotide molecules, wherein the interrogatable nucleotide molecules are designed to allow identification of the DNA methylation status of one or more imprint control regions (ICRs) that regulate one or more genes subject to monoalleleic expression in subjects.

### CROSS-REFERENCE TO RELATED APPLICATION

This application claims the benefit of U.S. Provisional Patent Application Ser. No. 63/050,086, filed Jul. 9, 2020, the disclosure of which is incorporated herein by reference in its entirety.

### GOVERNMENT INTEREST

This invention was made with government support under grant numbers HD093351, HD098857, MD011746, and ES025128 awarded by the National Institutes of Health. The government has certain rights in the invention.

### TECHNICAL FIELD

The subject matter described herein relates to the identification and analysis of differentially methylated allelic DNA sequences associated with regulating monoallelic expression of imprinted genes (i.e., Imprint Control Regions (ICRs)). More specifically, the subject matter relates to genetic arrays (e.g., gene chips) that can be used to determine imprinting at genetic loci subject to parent-of-origin monoallelic gene expression, and methods for using the arrays and the data generated therefrom.

### BACKGROUND

Epigenetic regulation is a mechanism by which gene expression is altered by DNA modifications that do not alter the base sequence of genomic DNA. Cellular differentiation during development is an epigenetic mechanism, by which cell-type specific genes are silenced as cell fate is determined. Epigenetics is also a means by which organisms respond to environmental exposures, allowing adaptive responses. Such changes, particularly those that occur in early development, can cause long-term expression changes in mechanistic pathways contributing to a broad range of clinically important outcomes, including neurological disorders (Lorgen-Ritchie et al., 2019), cardio- and cerebrovascular diseases (Jirtle, 2004; Jirtle & Skinner, 2007), cancer (Hoyo et al., 2009; Pigeyre et al., 2016) and their major risk factors, including obesity (e.g. metabolism, nutrient acquisition, fat deposition, appetite, and satiety; Franks & McCarthy, 2016; Pigeyre et al., 2016). Both covalent DNA methylation at cytosines of CpG dinucleotides and histone modifications are epigenetic modifications known to regulate chromatin structure and gene expression. The stability of the DNA molecule has made DNA methylation a highly utilized marker, measurable from cell sources regardless of preservation (e.g., fresh, frozen, FFPE) by bisulfite sequencing using targeted and high-throughput applications.

However, cell-type specific DNA methylation, and ongoing environmentally-induced responsive changes complicate the use of DNA methylation in human case/control or cross-sectional exposed/non-exposed studies. In such studies, DNA methylation measured in peripheral cell types, accessible from otherwise healthy individuals, is not necessarily representative of the methylation in inaccessible cell types, tissues and organs involved in neurological and metabolic diseases. Additionally, these complex diseases themselves are capable of altering epigenetic marks, and this temporal ambiguity between methylation marks and disease complicates causal inference.

Methylation marks that control imprinted genes offer a unique opportunity to overcome these shortcomings, especially for assessing outcomes from early life exposures. The defining feature of genomic imprinting is monoallelic gene expression, regulated by differentially methylated regions (DMRs) that are defined by reciprocal methylation status of the parental alleles; these regulatory DMRs are referred to as imprint control regions (ICRs). Epi-mutations (e.g., aberrant methylation) of ICRs are associated with growth and nutrient acquisition (reviewed in Cassidy & Charalambous, 2018). Because an important role of genomic imprinting is also to control gene dosage, methylation marks are similar across individuals. Unlike methylation levels which naturally diverge with cell differentiation and aging, these methylation marks are established prior to germ-layer specification, and are maintained in somatic tissues throughout life (Murphy, 2012). Thus, similarity across all tissues allows ascertainment of ICR methylation in peripheral cells as a proxy for normally inaccessible tissues. Moreover, stability with age makes these marks long-term records of early exposures. Imprinted genes are estimated to comprise 1-6% of the human genome (Luedi et al., 2007). These genes are over-selected for growth regulators, are critical in early embryonic development (Waterland, 2003) and altered expression results in growth disorders (Kitsiou-Tzeli & Tzetis, 2017). However, only 24 ICRs regulating approximately 120 imprinted genes are currently known (Skaar et al., 2012; Bernal et al., 2013).

Among diseases for which early detection could decrease risk of progression is Alzheimer's Disease (AD), a neuro-degenerative condition whose prevalence is on the increase. In the US, the annual costs of AD already exceed $280 billion, including $186 billion in Medicare and Medicaid payments, and estimates suggest that early diagnosis of Alzheimer's disease could save the nation ˜$7 trillion in long-term health care costs according to estimates from the Alzheimer's Association. Multiple lines of evidence suggest that multiple prenatal exposures contribute to AD risk, that susceptibility can be connected to early childhood risk factors (Seifan et al., 2015), and that performance on cognitive measures in early adulthood are predictive of Alzheimer risk (Snowdon et al., 1996). Related to such data and significant for this work, a handful of ICRs regulating known imprinted genes have been associated with AD risk (Lorgen-Ritchie et al., 2019). Two other psychiatric conditions with developmental origins are autism and schizophrenia, considered two sides of the same coin, as they are reciprocal regarding brain overdevelopment (autism) and underdevelopment (schizophrenia). It has been theorized that the origins of these two disorders lie in dysregulation of imprinted genes, with their alternative roles in promoting and restricting early growth and development (Crespi, 2008), and imprinted genes and regions have been connected to these conditions (Li et al., 2020), Such sequence regions could be developed into early risk markers diseases, disorders, and/or conditions including but not limited to those associated with neurological diseases, disorders, and/or conditions, but as yet, remain uncharacterized.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to methods for determining an imprinting status of a gene subject to parent-of-origin, monoalleleic expression in a subject. In some embodiments, the methods comprise (a) providing a nucleic acid preparation isolated from a cell, tissue, or organ of the subject, wherein the nucleic acid preparation comprises genomic DNA sequences derived from both alleles of the gene and that correspond to one or more Imprint Control Regions (ICRs) selected from the group consisting of ICRs 1-1611 as disclosed herein and/or the genomic regions associated with SEQ ID NOs: 1612-1816; and (b) identifying in the nucleic acid preparation the degree of and/or locations of methylation of both alleles of the gene with respect to the one or more ICRs and/or the genomic regions associated with SEQ ID NOs: 1612-1816, whereby an imprinting status of the gene in the subject is identified. In some embodiments, the subject is a human. In some embodiments, the genomic DNA sequences correspond to at least 100, 250, 500, 1000, or all of ICRs 1-1611 and/or the genomic regions associated with SEQ ID NOs: 1612-1816. In some embodiments, the identifying comprises interrogating a nucleic acid array that comprises nucleic acids that correspond to ICRs 1-1611 and/or the genomic regions associated with SEQ ID NOs: 1612-1816 and/or an informative subset thereof. In some embodiments, the interrogating comprises hybridizing bisulfite converted genomic DNA present in the nucleic acid preparation to a plurality of target probes, wherein the plurality of target probes correspond to the ICRs 1-1611 and/or the genomic regions associated with SEQ ID NOs: 1612-1816 and/or the informative subset thereof. In some embodiments, the interrogating comprises (a) hybridizing genomic DNA present in the nucleic acid preparation subsequent to a bisulfite converting treatment to the plurality of target probes present on a solid support, and further wherein the solid support comprises, consists essentially of, or consists of (i) target probes that comprise, consist essentially of, or consist of nucleotide sequences that are complementary to the nucleic acid sequences of ICRs 1-1611 and/or the genomic regions associated with SEQ ID NOs: 1612-1816 and/or the informative subset of prior to the bisulfite converting treatment; and (ii) target probes that comprise, consist essentially of, or consist of nucleotide sequences that differ from (i) above and are only complementary to the nucleic acid sequences of ICRs 1-1611 and/or the genomic regions associated with SEQ ID NOs: 1612-1816 and/or the informative subset of subsequent to the bisulfite converting treatment; and (c) calculating a methylation fraction of the genomic DNA present in the nucleic acid preparation by determining a ratio of hybridization of the target probes of (a) to the target probes of (b), wherein the ratio of hybridization provides a measure of the methylation fraction.

In some embodiments, the presently disclosed subject matter also relates to methods for detecting a presence of and/or a susceptibility to a medical condition associated with monoallelic gene expression in a subject. In some embodiments, the methods comprise (a) obtaining a biological sample from the subject, wherein the biological sample comprises one or more nucleic acid molecules that correspond to an Imprint Control Region (ICR) selected from the group consisting of ICRs 1-1611 and/or the genomic regions associated with SEQ ID NOs: 1612-1816 and/or a subset thereof; (b) analyzing the one or more nucleic acid molecules to determine the DNA methylation status of one or both alleles of at least one imprinted gene associated with at least one of ICRs 1-1611 and/or the genomic regions associated with SEQ ID NOs: 1612-1816; and (c) comparing the DNA methylation status of the one or both alleles of the at least one imprinted gene associated with the at least one of ICRs 1-1611 and/or the genomic regions associated with SEQ ID NOs: 1612-1816 to a control DNA methylation status, wherein the comparing detects a presence of and/or a susceptibility to a medical condition associated with monoallelic gene expression in a subject. In some embodiments, the DNA methylation status comprises one or more epigenomic features of at least one imprinted gene. In some embodiments, the one or more epigenomic features comprises a methylation profile of the subject with respect to at least one imprinted gene. In some embodiments, the one or more epigenomic features are selected from the group consisting of a DNA sequence methylation state, a nucleosome positioning feature, and a histone modification. In some embodiments, the one or more epigenomic features relates to a gene for which expression or lack of expression is associated with the medical condition. In some embodiments, the medical condition is Alzheimer's disease, autism, and schizophrenia. In some embodiments, at least one imprinted gene is selected from the set of genes associated with Alzheimer's disease (AD), where in the set of genes is identified as being associated with AD based on proximity to epigenomic features, correlated expression in association to epigenomic features, or reported association to Alzheimer disease in combination with either of the first two criteria. In some embodiments, the biological sample comprises genomic DNA isolated from a cell, tissue, or organ of the subject, optionally a cell, tissue, or organ that is not affected by the medical condition.

In some embodiments, the presently disclosed subject matter also relates to methods for predicting susceptibility to future development of a medical condition associated with monoallelic expression in a subject prior to the onset of any symptoms of the medical condition in the subject. In some embodiments, the methods comprise (a) a biological sample from the subject, wherein the biological sample comprises one or more nucleic acid molecules that correspond to an Imprint Control Region (ICR) selected from the group consisting of ICRs 1-1611 and/or the genomic regions associated with SEQ ID NOs: 1612-1816 and/or a subset thereof; (b) analyzing the one or more nucleic acid molecules to determine the DNA methylation status of one or both alleles of at least one imprinted gene associated with at least one of ICRs 1-1611 and/or the genomic regions associated with SEQ ID NOs: 1612-1816; and (c) determining whether the DNA methylation status determined correlates with future development of the medical condition, whereby a susceptibility to future development of the medical condition is predicted. In some embodiments, the DNA methylation status comprises one or more epigenomic features of at least one imprinted gene. In some embodiments, one or more epigenomic features comprises a methylation profile of the subject with respect to at least one imprinted gene. In some embodiments, one or more epigenomic features are selected from the group consisting of a DNA sequence methylation state, a nucleosome positioning feature, and a histone modification. In some embodiments, the one or more epigenomic features relates to a gene for which expression or lack of expression is associated with the medical condition. In some embodiments, the medical condition is Alzheimer's disease, autism, schizophrenia, and/or a malignancy, which in some embodiments can be hepatocellular carcinoma. In some embodiments, the at least one imprinted gene is a gene as disclosed herein. In some embodiments, the biological sample comprises genomic DNA isolated from any cell, tissue, or organ of the subject, including a cell, tissue, or organ that is generally unaffected in subjects who have the medical condition, and not necessarily usable for diagnosis of conditions affecting target tissues by means specific to those affected tissues, including, but not limited to, physical morphology, immunological assays, protein expression, or RNA expression.

In some embodiments, the presently disclosed subject matter also relates to nucleic acid arrays comprising one or more interrogatable nucleotide molecules, wherein the interrogatable nucleotide molecules are designed to allow identification of the DNA methylation status of ICRs that regulate one or more genes subject to monoalleleic expression in a biological sample isolated from a subject. In some embodiments, the nucleic acid array comprises, consists essentially of, or consists of a plurality of interrogatable nucleotide molecules that correspond to one or more of ICRs 1-1611 and/or the genomic regions associated with SEQ ID NOs: 1612-1816, optionally wherein the interrogatable nucleotide molecules comprise, consist essentially of, or consist of (a) target probes that comprise, consist essentially of, or consist of nucleotide sequences that are complementary to the nucleic acid sequences of ICRs 1-1611 and/or the genomic regions associated with SEQ ID NOs: 1612-1816 and/or an informative subset thereof; and (b) target probes that comprise, consist essentially of, or consist of nucleotide sequences that are complementary to the nucleic acid sequences of ICRs 1-1611 and/or the genomic regions associated with SEQ ID NOs: 1612-1816 and/or the informative subset thereof subsequent to exposing the nucleic acid sequences of ICRs 1-1611 and/or the genomic regions associated with SEQ ID NOs: 1612-1816 and/or the informative subset thereof to a bisulfite converting treatment. In some embodiments, the interrogatable nucleotide molecules can be interrogated with human genomic DNA. In some embodiments, the plurality of interrogatable nucleotide molecules correspond to at least 100, 250, 500, 1000, or all of ICRs 1-1611 and/or the genomic regions associated with SEQ ID NOs: 1612-1816.

Accordingly, it is an object of the presently disclosed subject matter to provide compositions and methods for assessing differentially methylated DNA sequences associated with monoallelic gene expression and disease.

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

FIGS. 1A-1E. Detection of ICRs based upon genome-wide DNA methylation sequences from conceptal kidney, liver, and brain, as well as gametes. FIG. 1A is a graph showing the coverage (number of reads per base pair) for brain, kidney, liver, sperm, and oocytes. Putative ICRs are identified based on consecutive CpGs with allele specific differential methylation in a specified range. Narrowing of the methylation window centered on 50% reduces the number of candidates (FIG. 1B) but continues to identify the majority of known ICRs (FIG. 1C). The size range of candidates is similar with known ICRs (FIGS. 1D and 1E). For many of the known ICRs, overlapping candidate ICRs extend beyond the current definitions.

FIGS. 2A-2D. Example of known and novel putative ICRs. MEG3 and PEG10 have associated known ICR loci (FIGS. 2A and 2B), and in both cases are overlapped by candidate ICRs that extend beyond the current definitions. Using the identification criteria, novel ICRs were detected for IGF2R and KCNQ1OT1 (FIGS. 2C and 2D). IGF2R is imprinted in some mammals, but does not have consistently observed imprinted expression in humans.

FIGS. 3A-3F. Identification of Alzheimer's Disease (AD) DMRs and overlap with ICRs. Using DNA from AD cases and controls, for both African American (AA) and European American (EA) patients, DNA regions with differential methylation between cases and controls were identified. An excellent bisulfite conversion rate was attained in all cases (FIG. 3A). Moreover, the coverage range was between 15×-36× (FIGS. 3B and 3C) with no sequence duplication bias (FIG. 3D). The total DMRs detected from cases and controls from EA and AA groups separately and combined, and the overlap were shown (FIG. 3D). In the case of the EA samples, patient blood was available for comparison with matching controls to generate DMRs, which were intersected with the DMRs generated from AD patient brain tissue (FIGS. 3E and 3F).

FIGS. 4A-4C. Overlap of a putative ICR overlapping an AD DMR in AAs and EAs. Overlap of AD DMRs with 1495 ICRs (FIG. 4A). An AD case-control comparison identified a DMR mapping to AKAP2, which overlaps an ICR identified from conceptal tissues and gametes. (FIG. 4B). There are also a set of regions in the intersection between AD brain DMRs, AD blood DMRs, and ICRs. (FIG. 4C).

FIG. 5. Workflow to identify putative ICRs. FIG. 5 shows an exemplary workflow for identifying putative ICRs.

FIG. 6. Venn diagram illustrating DMR to ICR mapping results. AA: African American, W: White, AD: Alzheimer's Disease.

### BRIEF DESCRIPTION OF THE SEQUENCE LISTING

SEQ ID NOs: 1-1611 are the nucleotide sequences of imprint control regions (ICRs) 1-1611 present in the human genome. SEQ ID NOs: 1-1611 correspond to ICR_1 through ICR_1611, respectively.

SEQ ID NOs: 1612-1816 are the nucleotide sequences of human genomic sequences that were identified in whole genome methylation analyses in Alzheimer's patients but that did not align with any of the ICRs corresponding to SEQ ID NOs: 1-1611.

### DETAILED DESCRIPTION

Disclosed herein is the identification of human ICRs through methyl-sequencing of fetal tissues representing the three germ layers, the endoderm, mesoderm, and ectoderm, as well as using methyl-sequence from gametes. Also disclosed herein are assessments of the similarities of methylation marks of ICRs in accessible cell types including mixed leukocytes, monocytes, and human umbilical vein endothelial cells (HUVECs). Using frontal cortex-derived DNA, it is shown that aberrant methylation of a sizable proportion of ICRs was found in Alzheimer's disease but not control brains.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” mean “one or more” when used in this application, including the claims. Thus, the phrase “a cell” refers to one or more cells, unless the context clearly indicates otherwise.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include the use of either of the other two terms. For example, it is understood that the methods of the presently disclosed subject matter in some embodiments comprise the steps that are disclosed herein and/or that are recited in the claims, in some embodiments consist essentially of the steps that are disclosed herein and/or that are recited in the claims, and in some embodiments consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, the presently disclosed subject matter relates to human subjects.

Similarly, all genes, gene names, and gene products disclosed herein are intended to correspond to orthologs from any species for which the compositions and methods disclosed herein are applicable. Thus, the terms include, but are not limited to genes and gene products from humans. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are also intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about,” as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods and/or to employ the presently disclosed arrays.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

As used herein, the phrase “derived from” refers to an entity that is present either in another entity and/or in some embodiments in the same entity but in a different context. In terms of biological samples and nucleic acids, the phrase “derived from” can be synonymous with “isolated from”. However, especially in the case of a biological molecule, the phrase “derived from” can also refer to the fact that the biological molecule is present in a different context or form in one situation versus another. For example, in some embodiments, the presently disclosed methods employ nucleic acid molecules “derived from” a gene (e.g., a gene listed in any of the Tables disclosed herein). In this context, it is understood that a nucleic acid molecule is “derived from” a gene if the nucleic acid molecule can be generated naturally or artificially by employing genetic and/or epigenomic information that is associated with the gene in the subject. In some embodiments, a nucleic acid molecule is “derived from” a gene if it is encoded by the gene, is a transcription product of the gene, or otherwise is generated based on genetic or non-genetic information that is provided by the gene.

As used herein, the term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an imprint regulatory element, an enhancer, an origin of replication, a 5′ or 3′ untranslated region, a coding region, and/or a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc.

As used herein, the term “gene” is used broadly to refer to any segment of DNA associated with a biological function. Thus, genes include, but are not limited to, coding sequences, the regulatory sequences required for their expression (e.g., 5′ regulator sequences, 3′ regulatory sequences, and combinations thereof), intron sequences associated with the coding sequences, and combinations thereof. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The phrase “hybridizing specifically to” refers to the binding, duplexing, or hybridizing of a molecule only to a particular nucleotide sequence under stringent conditions when that sequence is present in a complex mixture (e.g., total cellular) of DNA and/or RNA. The phrase “bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, the term “isolated”, when used in the context of an isolated nucleic acid or an isolated polypeptide, is a nucleic acid or polypeptide that, by the hand of man, exists apart from its native environment and is therefore not a product of nature. An isolated nucleic acid molecule or polypeptide can exist in a purified form or can exist in a non-native environment such as, for example, in a transformed host cell.

As used herein, the term “native” refers to a gene that is naturally present in the genome of an untransformed cell. Similarly, when used in the context of a polypeptide, a “native polypeptide” is a polypeptide that is encoded by a native gene of an untransformed cell's genome. Thus, the terms “native” and “endogenous” are synonymous.

As used herein, the term “naturally occurring” refers to an object that is found in nature as distinct from being artificially produced or manipulated by man. For example, a polypeptide or nucleotide sequence that is present in an organism (including a virus) in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

As used herein, the term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Ohtsuka et al., 1985; Batzer et al., 1991; Rossolini et al., 1994). The terms “nucleic acid” or “nucleic acid sequence” can also be used interchangeably with gene, cDNA, and mRNA encoded by a gene.

As used herein, the phrase “oligonucleotide” refers to a polymer of nucleotides of any length. In some embodiments, an oligonucleotide is a primer that is used in a polymerase chain reaction (PCR) and/or reverse transcription-polymerase chain reaction (RT-PCR), and the length of the oligonucleotide is typically between about 15 and 30 nucleotides. In some embodiments, the oligonucleotide is present on an array and is specific for a gene of interest. In whatever embodiment that an oligonucleotide is employed, one of ordinary skill in the art is capable of designing the oligonucleotide to be of sufficient length and sequence to be specific for the gene of interest (i.e., that would be expected to specifically bind only to a product of the gene of interest under a given hybridization condition).

As used herein, the phrase “percent identical”, in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection. See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the term “subject” refers to any organism for which analysis of gene expression would be desirable. Thus, the term “subject” is desirably a human subject, although it is to be understood that the principles of the presently disclosed subject matter indicate that the presently disclosed subject matter is effective with respect to invertebrate and to all vertebrate species, including Therian mammals (e.g., Marsupials and Eutherians), which are intended to be included in the term “subject”. Moreover, a mammal is understood to include any mammalian species in which detection of differential gene expression is desirable, particularly agricultural and domestic mammalian species. The methods of the presently disclosed subject matter are particularly useful in the analysis of gene expression in warm-blooded vertebrates, e.g., mammals.

More particularly, the presently disclosed subject matter can be used for assessing imprinting and its consequences in a mammal such as a human. Also provided is the analysis of gene expression in mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses (e.g., thoroughbreds and race horses).

Additionally, in some embodiments the term “subject” refers to a biological sample as defined herein, which includes but is not limited to a cell, tissue, or organ that is isolated from an organism. Thus, it is understood that the methods and compositions disclosed herein can be employed for assessing imprinting and its consequences in a subject that is an organism but can also be employed for assessing imprinting and its consequences in a subject that is a biological sample isolated from an organism. Accordingly, the methods and compositions disclosed herein are intended to be applicable to assessing imprinting and its consequences in vivo as well as in vitro.

### II. Compositions Including Interrogatable Nucleic Acid Arrays

In some embodiments, the presently disclosed subject matter relates to nucleic acid arrays comprising, consisting essentially of, or consisting of one or more interrogatable nucleotide molecules, wherein the interrogatable nucleotide molecules are designed to allow identification of the DNA methylation status of one or more of ICRs that regulate one or more genes subject to monoalleleic expression in a biological sample isolated from a subject. In some embodiments, the one or more ICRs are selected from the group consisting of ICRs 1-1611 as defined herein.

Methods for producing nucleic acid arrays are known, and each of these can be employed to generate a nucleic acid array for use in the presently disclosed subject matter. Exemplary patents and patent applications that disclose nucleic acids arrays and their production include U.S. Patent Application Publication Nos. 2010/0056397, 2010/0304997, 2011/0105357, 2015/0232921 and U.S. Pat. Nos. 6,355,431; 6,429,027; 6,936,461; 7,824,917; 9,395,360; and 9,828,640, each of which is incorporated by reference herein in its entirety. See also Pirrung, 2002; Sandoval et al., 2011; Moran et al., 2016; and Krämer et al., 2019, each of which is incorporated by reference herein in its entirety.

In some embodiments, a nucleic acid array of the presently disclosed subject matter comprises, consists essentially of, or consists of a plurality of interrogatable nucleotide molecules that correspond to one or more of ICRs 1-1611. As used herein the phrase “interrogatable nucleotide molecules” refers to nucleic acids that are present on the nucleic acid array that can be used to determine the presence or absence of nucleic acids in a biological sample, which typically occurs by DNA-DNA hybridization. As such, in some embodiments the interrogatable nucleic acids present on the nucleic acid array are partially or completely single stranded such that single stranded regions of nucleic acids present in a biological sample can be hybridized thereto under various hybridization conditions (e.g., stringency conditions).

In the context of the nucleic acid arrays of the presently disclosed subject matter that can in some embodiments be employed to detect differential methylation of genomic DNA in a biological sample, in some embodiments the interrogatable nucleotide molecules comprise, consist essentially of, or consist of (a) target probes that comprise, consist essentially of, or consist of nucleotide sequences that are complementary to the nucleic acid sequences of ICRs 1-1611 or an informative subset thereof; and (b) target probes that comprise, consist essentially of, or consist of nucleotide sequences differ from (a) above and that are complementary to the nucleic acid sequences of ICRs 1-1611 or the informative subset thereof only subsequent to exposing the nucleic acid sequences of ICRs 1-1611 or the informative subset thereof to a bisulfite converting treatment.

As would be understood by one of ordinary skill in the art, methylation of genomic DNA at the C5 position of cytosines within CpG dinucleotides is associated with various epigenetic phenomena including, but not limited to gene expression, regulation of development, cellular proliferation and differentiation, and chromosome stability. The technique known as bisulfite sequencing takes advantage of the fact that cytosine can be reacted with sodium bisulfite, which deaminates the cytosine to a uracil molecule. C5-methylated cytosine, on the other hand, is insensitive to this reaction. As a consequence, single stranded DNA molecules that have been treated with sodium bisulfite will contain uracil nucleotides in place of unmethylated cytosines, and thus will hybridize to single stranded nucleic acids that have adenine in the corresponding location. In contrast, single stranded DNA molecules that have been treated with sodium bisulfite will retain unreacted C5-methyl cytosines, and thus will only hybridize to single stranded nucleic acids that have guanine in the corresponding location. As such, the presence or absence of cytosine methylation can be determined in DNA samples by treating said DNA samples with bisulfite followed by a relative quantification of hybridization of those treated molecules to single stranded nucleic acids that would be expected to hybridize only to DNA strands with uracils where (unmodified) cytosines had been present as compared to hybridization of treated molecules to single stranded nucleic acids that would be expected to hybridize to DNA strands with C5-methylated cytosines. In the latter case (i.e., to assay the presence of C5-methylated cytosine), those single stranded nucleic acids would be the exact reverse complements of the genomic DNA: i.e., would have guanines “across from” the C5-methylated cytosine in a double stranded molecule. In the case of unmethylated cytosines, however, the single stranded nucleic acids to which these molecules would hybridize would be the reverse complement of the genomic DNA but with an adenine rather than a guanine present in each location “across from” where an unmodified cytosine is present in the genomic DNA.

Therefore, with respect to the presently disclosed subject matter, the target probes that comprise, consist essentially of, or consist of nucleotide sequences that are complementary to the nucleic acid sequences of ICRs 1-1611 or an informative subset thereof of (a) above would be used to detect sites of C5 methylation of cytosine and the target probes that comprise, consist essentially of, or consist of nucleotide sequences that are different from (a) and are complementary only to the nucleic acid sequences of ICRs 1-1611 or the informative subset thereof subsequent to exposing the nucleic acid sequences of ICRs 1-1611 or the informative subset thereof to a bisulfite converting treatment of (b) above would be used to detect cytosines that are not C5 methylated.

Thus, in some embodiments the nucleic acid arrays of the presently disclosed subject matter include interrogatable nucleotide molecules that function as pairs: one member of the pair corresponding to a molecule that hybridizes to one or more of ICRs 1-1611 assuming the presence of one or more C5-methylated cytosines, and a second member of the pair including the same nucleotide sequence by with one or more guanosines replaced with adenosines.

In some embodiments, the interrogatable nucleotide molecules can be interrogated with human genomic DNA.

It is understood that the nucleic acid arrays of the presently disclosed subject matter need not include interrogatable nucleotide molecules that correspond to each and every one of ICRs 1-1611, however in some embodiments the presently disclosed subject matter encompasses interrogatable nucleotide molecules that correspond to each and every one of ICRs 1-1611. Accordingly, in some embodiments the nucleic acid arrays of the presently disclosed subject matter comprise, consist essentially of, or consist of interrogatable molecules that correspond to a subset of ICRs 1-1611. Any subset of ICRs 1-1611 can be included on a nucleic acid array of the presently disclosed subject matter. By way of example and not limitation, a nucleic acid array of the presently disclosed subject matter can include at least 5, 10, 25, 50, 100, 250, 500, 1000, or all of ICRs 1-1611, or any whole number between 1 and 1495.

In some embodiments, a nucleic acid array is designed for a specific application, for which particular subsets of ICRs 1-1611 might be desired while others would not be relevant. Such a particular subset that is relevant to a particular application is referred to herein as an “informative subset” of ICRs 1-1611. The precise nature of the “informative subset” can in some embodiments differ among various applications, and thus an “informative subset” is whatever subset of ICRs one might which to interrogate with respect to a particular disease, disorder, condition, or detection protocol.

### III. Methods

As set forth herein, the compositions (in some embodiments, the nucleic acid arrays) of the presently disclosed subject matter can be used to identify differences in DNA methylation of any DNA sample from any biological sample. The results of these identifications can be used for various purposes, including but not limited to those explicitly disclosed herein.

III.A. Methods for Identifying Imprinted Statuses of Genes in Subjects

For example, in some embodiments the presently disclosed subject matter relates to methods for determining an imprinting status of a gene or of a plurality of genes that is/are subject to parent-of-origin, monoalleleic expression in a subject. In some embodiments, the methods comprise, consist essentially of, or consist of providing a nucleic acid preparation isolated from a cell, tissue, or organ of the subject, wherein the nucleic acid preparation comprises genomic DNA sequences derived from both alleles of the gene and that correspond to one or more Imprint Control Regions (ICRs) selected from the group consisting of ICRs 1-1611 as disclosed herein; and identifying in the nucleic acid preparation the degree of and/or locations of methylation of both alleles of the gene with respect to the one or more ICRs, whereby an imprinting status of the gene in the subject is identified. In some embodiments, the subject is a human.

As used herein, the phrase “imprinting status” refers to a summary of one or more locations in a genomic DNA sample that are characterized by the presence or absence of C5-methylated cytosines. Thus, in some embodiments an imprinting status is a profile of the presence or absence of one or more C5-methylated cytosines at one or more locations. In some embodiments, an imprinting status is a profile of the presence of absence of a plurality of, in some embodiments, all C5-methylated cytosines at one or more locations. The imprinting status of a subject at a given time can be compared to a control. If the relevant inquiry with respect to a subject's imprinting status relates to that subject's imprinting status as compared to a population, then the subject's imprinting status can be compared to an imprinting status of the population. Because individual members of a population can differ in their individual imprinting statuses, a population's imprinting status to be employed as a control can in some embodiments be a summary of the most common C5-methylation patterns at individual locations, even though certain individuals in the population can differ from what is considered the population's imprinting status/profile.

Thus, in some embodiments a method for identifying an imprinted status of a gene subject to monoalleleic expression in a subject can comprise, consist essentially of, or consist of (a) hybridizing genomic DNA present in the nucleic acid preparation subsequent to a bisulfite converting treatment to the plurality of target probes present on a solid support, and further wherein the solid support comprises, consists essentially of, or consists of (i) target probes that comprise, consist essentially of, or consist of nucleotide sequences that are complementary to the nucleic acid sequences of ICRs 1-1611 or the informative subset of prior to the bisulfite converting treatment; (ii) target probes that comprise, consist essentially of, or consist of nucleotide sequences that are complementary to the nucleic acid sequences of ICRs 1-1611 or the informative subset of subsequent to the bisulfite converting treatment; and (c) calculating a methylation fraction of the genomic DNA present in the nucleic acid preparation by determining a ratio of hybridization of the target probes of (a) to the target probes of (b), wherein the ratio of hybridization provides a measure of the methylation fraction.

III.B. Methods for Detecting Presence of and/or Susceptibility to Medical Conditions Associated with Monoallelic Gene Expression in Subjects

In some embodiments, the presently disclosed subject matter also relates to methods for detecting a presence of and/or a susceptibility to a medical condition associated with monoallelic gene expression in a subject. Generally, the methods relate to identifying informative DNA methylation differences in a subject's DNA that are predictive of the presence of and/or a susceptibility to a medical condition associated with monoallelic gene expression. In some embodiments, the methods comprise (a) obtaining a biological sample from the subject, wherein the biological sample comprises one or more nucleic acid molecules that correspond to an Imprint Control Region (ICR) selected from the group consisting of ICRs 1-1611 or a subset thereof; (b) analyzing the one or more nucleic acid molecules to determine the DNA methylation status of one or both alleles of at least one imprinted gene associated with at least one of ICRs 1-1611; and (c) comparing the DNA methylation status of the one or both alleles of the at least one imprinted gene associated with the at least one of ICRs 1-1611 to a control DNA methylation status, wherein the comparing detects a presence of and/or a susceptibility to a medical condition associated with monoallelic gene expression in a subject. It is known in the art that certain medical conditions (e.g., a disease, disorder, or condition associated with a medical diagnosis) are associated with monoalleleic gene expression. Examples of such medical conditions include Prader_Willi syndrome (related to a cluster of genes on Chr 15:q11-13, including SNRPN, NDN, and multiple small nucleolar RNAs (SNORDs) in particular, and ICRs with coordinates/nucleotide positions on chromosome 15 of 23,647,239-23,648,622; 23,686,523-23,686,574; 23,758,904-23,759,277; 23,860,773-23,861,094; 23,897,271-23,897,645; 24,877,837-24,878,217; 24,954,592-24,956,828 of the human chromosome sequence set forth in Accession No. NC 000015.10 of the GENBANK® biosequence database), Angelman syndrome (related to the same Chr15:q11-q13 region as Prader-Willi syndrome, including UBE3A in particular, and the same ICRs), Silver-Russel syndrome and male infertility (related to H19 and IGF2, and ICRs on chromosome 11 with coordinates/nucleotide positions on chromosome 11 of 1,997,886-1,999,417; 1,999,793-2,000,383; 2,000,487-2,001,247; and 2,001,655-2,003,118 of the human chromosome sequence set forth in Accession No. NC 000011.10 of the GENBANK® biosequence database), Beckwith_Wiedemann syndrome (related to CDKN1C), Birk-Barel syndrome (related to KCNK9), preeclampsia and split hand-foot malformation (related to DLXS), retinoblastoma (related to RB1, and ICRs on chromosome 13 with coordinates/nucleotide positions 48,317,373-48,317,679 and 48,317,894-48,321,417 of the human chromosome sequence set forth in Accession No. NC_000013.11 of the GENBANK® biosequence database), breast and ovarian cancer (related to DIRAS3, on chromosome 1, coordinates/nucleotide positions 68,046,822-68,047,535; 68,049,858-68,051,097; and 68,051,239-68,051,861 of the human chromosome sequence set forth in Accession No. NC 000001.11 of the GENBANK® biosequence database). All coordinates given are from human genome build 38, which correspond to the Accession NOs: summarized in Table 1.

TABLE 1
Accession NOs. for Human Chromosomal Sequences in
the GENBANK ® Biosequence Database

| | | | |
|---|---|---|---|
| Human Chromosome 1 | NC_000001.11 | Human Chromosome 2 | NC_000002.12 |
| Human Chromosome 3 | NC_000003.12 | Human Chromosome 4 | NC_000004.12 |
| Human Chromosome 5 | NC_000005.10 | Human Chromosome 6 | NC_000006.12 |
| Human Chromosome 7 | NC_000007.14 | Human Chromosome 8 | NC_000008.11 |
| Human Chromosome 9 | NC_000009.12 | Human Chromosome 10 | NC_000010.11 |
| Human Chromosome 11 | NC_000011.10 | Human Chromosome 12 | NC_000012.12 |
| Human Chromosome 13 | NC_000013.11 | Human Chromosome 14 | NC_000014.9 |
| Human Chromosome 15 | NC_000015.10 | Human Chromosome 16 | NC_000016.10 |
| Human Chromosome 17 | NC_000017.11 | Human Chromosome 18 | NC_000018.10 |
| Human Chromosome 19 | NC_000019.10 | Human Chromosome 20 | NC_000020.11 |
| Human Chromosome 21 | NC_000021.9 | Human Chromosome 22 | NC_000022.11 |
| Human Chromosome X | NC_000023.11 | Human Chromosome Y | NC_000024.10 |

Another medical condition that is associated with the expression of particular genes, some of which have been shown to be associated by monoallelic expression, is autism. 1,904 associated genes have been reported (1,875 from the ENSEMBL hg38; see also Li et al., 2020). 82 of these autism-associated genes are within 10 kb of an ICR, and are summarized in Table 2.

TABLE 2
Exemplary Genetic Loci Associated with Autism and that are Within 10 kb of an ICR

| | | Ensembl Gene | Human | | |
|---|---|---|---|---|---|
| Symbol | ICR No.^{a} | Accession No. | Chromosome | Start^{b} | End^{b} |
| SAMD11 | ICR_5 | ENSG00000187634 | 1 | 923,928 | 944,581 |
| B3GALT6 | ICR_7 | ENSG00000176022 | 1 | 1,232,265 | 1,235,041 |
| LAMB3 | ICR_82 | ENSG00000196878 | 1 | 209,614,870 | 209,652,466 |
| SRD5A2 | ICR_636 | ENSG00000277893 | 2 | 31,522,480 | 31,581,067 |
| RUVBL1 | ICR_978 | ENSG00000175792 | 3 | 128,064,778 | 128,153,914 |
| TERT | ICR_1063 | ENSG00000164362 | 5 | 1,253,147 | 1,295,069 |
| TERT | ICR_1062 | ENSG00000164362 | 5 | 1,253,147 | 1,295,069 |
| DHFR | ICR_1080 | ENSG00000228716 | 5 | 80,626,228 | 80,654,983 |
| NR2F1 | ICR_1082 | ENSG00000175745 | 5 | 93,583,337 | 93,594,615 |
| PCDHA1 | ICR_1089 | ENSG00000204970 | 5 | 140,786,136 | 141,012,347 |
| PCDHA1 | ICR_1090 | ENSG00000204970 | 5 | 140,786,136 | 141,012,347 |
| PCDHA1 | ICR_1091 | ENSG00000204970 | 5 | 140,786,136 | 141,012,347 |
| PCDHA1 | ICR_1092 | ENSG00000204970 | 5 | 140,786,136 | 141,012,347 |
| PCDHA1 | ICR_1093 | ENSG00000204970 | 5 | 140,786,136 | 141,012,347 |
| PCDHA1 | ICR_1094 | ENSG00000204970 | 5 | 140,786,136 | 141,012,347 |
| PCDHA1 | ICR_1095 | ENSG00000204970 | 5 | 140,786,136 | 141,012,347 |
| PCDHA1 | ICR_1096 | ENSG00000204970 | 5 | 140,786,136 | 141,012,347 |
| PCDHA2 | ICR_1090 | ENSG00000204969 | 5 | 140,794,852 | 141,012,344 |
| PCDHA2 | ICR_1091 | ENSG00000204969 | 5 | 140,794,852 | 141,012,344 |
| PCDHA2 | ICR_1092 | ENSG00000204969 | 5 | 140,794,852 | 141,012,344 |
| PCDHA2 | ICR_1093 | ENSG00000204969 | 5 | 140,794,852 | 141,012,344 |
| PCDHA2 | ICR_1094 | ENSG00000204969 | 5 | 140,794,852 | 141,012,344 |
| PCDHA2 | ICR_1095 | ENSG00000204969 | 5 | 140,794,852 | 141,012,344 |
| PCDHA2 | ICR_1096 | ENSG00000204969 | 5 | 140,794,852 | 141,012,344 |
| PCDHA2 | ICR_1089 | ENSG00000204969 | 5 | 140,794,852 | 141,012,344 |
| PCDHA3 | ICR_1090 | ENSG00000255408 | 5 | 140,801,028 | 141,012,344 |
| PCDHA3 | ICR_1091 | ENSG00000255408 | 5 | 140,801,028 | 141,012,344 |
| PCDHA3 | ICR_1092 | ENSG00000255408 | 5 | 140,801,028 | 141,012,344 |
| PCDHA3 | ICR_1093 | ENSG00000255408 | 5 | 140,801,028 | 141,012,344 |
| PCDHA3 | ICR_1094 | ENSG00000255408 | 5 | 140,801,028 | 141,012,344 |
| PCDHA3 | ICR_1095 | ENSG00000255408 | 5 | 140,801,028 | 141,012,344 |
| PCDHA3 | ICR_1096 | ENSG00000255408 | 5 | 140,801,028 | 141,012,344 |
| PCDHA4 | ICR_1091 | ENSG00000204967 | 5 | 140,806,929 | 141,012,344 |
| PCDHA4 | ICR_1092 | ENSG00000204967 | 5 | 140,806,929 | 141,012,344 |
| PCDHA4 | ICR_1093 | ENSG00000204967 | 5 | 140,806,929 | 141,012,344 |
| PCDHA4 | ICR_1094 | ENSG00000204967 | 5 | 140,806,929 | 141,012,344 |
| PCDHA4 | ICR_1095 | ENSG00000204967 | 5 | 140,806,929 | 141,012,344 |
| PCDHA4 | ICR_1096 | ENSG00000204967 | 5 | 140,806,929 | 141,012,344 |
| PCDHA4 | ICR_1090 | ENSG00000204967 | 5 | 140,806,929 | 141,012,344 |
| PCDHA5 | ICR_1093 | ENSG00000204965 | 5 | 140,821,604 | 141,012,344 |
| PCDHA5 | ICR_1094 | ENSG00000204965 | 5 | 140,821,604 | 141,012,344 |
| PCDHA5 | ICR_1095 | ENSG00000204965 | 5 | 140,821,604 | 141,012,344 |
| PCDHA5 | ICR_1096 | ENSG00000204965 | 5 | 140,821,604 | 141,012,344 |
| PCDHA6 | ICR_1093 | ENSG00000081842 | 5 | 140,827,958 | 141,012,344 |
| PCDHA6 | ICR_1094 | ENSG00000081842 | 5 | 140,827,958 | 141,012,344 |
| PCDHA6 | ICR_1095 | ENSG00000081842 | 5 | 140,827,958 | 141,012,344 |
| PCDHA6 | ICR_1096 | ENSG00000081842 | 5 | 140,827,958 | 141,012,344 |
| PCDHA7 | ICR_1094 | ENSG00000204963 | 5 | 140,834,248 | 141,012,344 |
| PCDHA7 | ICR_1095 | ENSG00000204963 | 5 | 140,834,248 | 141,012,344 |
| PCDHA7 | ICR_1096 | ENSG00000204963 | 5 | 140,834,248 | 141,012,344 |
| PCDHA7 | ICR_1093 | ENSG00000204963 | 5 | 140,834,248 | 141,012,344 |
| PCDHA9 | ICR_1094 | ENSG00000204961 | 5 | 140,847,463 | 141,012,344 |
| PCDHA9 | ICR_1095 | ENSG00000204961 | 5 | 140,847,463 | 141,012,344 |
| PCDHA9 | ICR_1096 | ENSG00000204961 | 5 | 140,847,463 | 141,012,344 |
| PCDHA10 | ICR_1094 | ENSG00000250120 | 5 | 140,855,883 | 141,012,344 |
| PCDHA10 | ICR_1095 | ENSG00000250120 | 5 | 140,855,883 | 141,012,344 |
| PCDHA10 | ICR_1096 | ENSG00000250120 | 5 | 140,855,883 | 141,012,344 |
| PCDHA11 | ICR_1095 | ENSG00000249158 | 5 | 140,868,183 | 141,012,344 |
| PCDHA11 | ICR_1096 | ENSG00000249158 | 5 | 140,868,183 | 141,012,344 |
| PCDHA12 | ICR_1095 | ENSG00000251664 | 5 | 140,875,302 | 141,012,344 |
| PCDHA12 | ICR_1096 | ENSG00000251664 | 5 | 140,875,302 | 141,012,344 |
| PCDHA13 | ICR_1096 | ENSG00000239389 | 5 | 140,882,208 | 141,012,344 |
| PCDHA13 | ICR_1095 | ENSG00000239389 | 5 | 140,882,208 | 141,012,344 |
| ZNF311 | ICR_1143 | ENSG00000197935 | 6 | 28,994,785 | 29,005,316 |
| ZNF311 | ICR_1142 | ENSG00000197935 | 6 | 28,994,785 | 29,005,316 |
| DXO | ICR_1148 | ENSG00000204348 | 6 | 31,969,810 | 31,972,292 |
| DLL1 | ICR_1183 | ENSG00000198719 | 6 | 170,282,206 | 170,306,565 |
| FAM120B | ICR_1183 | ENSG00000112584 | 6 | 170,290,703 | 170,407,065 |
| GRB10 | ICR_1215 | ENSG00000106070 | 7 | 50,590,063 | 50,793,462 |
| LAMB1 | ICR_1240 | ENSG00000091136 | 7 | 107,923,799 | 108,003,255 |
| DPP6 | ICR_1251 | ENSG00000130226 | 7 | 153,887,097 | 154,894,285 |
| HTR5A | ICR_1252 | ENSG00000157219 | 7 | 155,070,324 | 155,085,749 |
| ARHGEF10 | ICR_1268 | ENSG00000104728 | 8 | 1,823,976 | 1,958,641 |
| SOX7 | ICR_1272 | ENSG00000171056 | 8 | 10,723,768 | 10,730,512 |
| EGR3 | ICR_1277 | ENSG00000179388 | 8 | 22,687,659 | 22,693,302 |
| CHRNA2 | ICR_1279 | ENSG00000120903 | 8 | 27,459,761 | 27,479,883 |
| CHRNA2 | ICR_1278 | ENSG00000120903 | 8 | 27,459,761 | 27,479,883 |
| PRKDC | ICR_1289 | ENSG00000253729 | 8 | 47,773,108 | 47,960,183 |
| KHDRBS3 | ICR_1308 | ENSG00000131773 | 8 | 135,457,457 | 135,656,722 |
| FOCAD | ICR_1321 | ENSG00000188352 | 9 | 20,658,309 | 20,995,955 |
| GSN | ICR_1372 | ENSG00000148180 | 9 | 121,207,794 | 121,332,843 |
| GRIN1 | ICR_1387 | ENSG00000176884 | 9 | 137,138,390 | 137,168,762 |
| CACNB2 | ICR_121 | ENSG00000165995 | 10 | 18,140,677 | 18,541,869 |
| PTCHD3 | ICR_124 | ENSG00000182077 | 10 | 27,398,187 | 27,414,368 |
| KIF11 | ICR_151 | ENSG00000138160 | 10 | 92,593,286 | 92,655,395 |
| EXOC6 | ICR_152 | ENSG00000138190 | 10 | 92,834,713 | 93,059,493 |
| EBF3 | ICR_169 | ENSG00000108001 | 10 | 129,835,283 | 129,963,841 |
| BRSK2 | ICR_196 | ENSG00000174672 | 11 | 1,389,899 | 1,462,689 |
| KCTD21 | ICR_224 | ENSG00000188997 | 11 | 78,171,249 | 78,188,822 |
| KRT83 | ICR_263 | ENSG00000170523 | 12 | 52,314,301 | 52,321,398 |
| PCCA | ICR_312 | ENSG00000175198 | 13 | 100,089,015 | 100,530,437 |
| ITPK1 | ICR_343 | ENSG00000100605 | 14 | 92,936,914 | 93,116,320 |
| C14orf2 | ICR_355 | ENSG00000156411 | 14 | 103,912,288 | 103,928,269 |
| MAGEL2 | ICR_368 | ENSG00000254585 | 15 | 23,643,544 | 23,647,841 |
| SNRPN | ICR_373 | ENSG00000128739 | 15 | 24,823,637 | 24,978,723 |
| SNRPN | ICR_374 | ENSG00000128739 | 15 | 24,823,637 | 24,978,723 |
| APBA2 | ICR_376 | ENSG00000034053 | 15 | 28,884,483 | 29,118,315 |
| C15orf62 | ICR_379 | ENSG00000188277 | 15 | 40,770,080 | 40,772,449 |
| TSC2 | ICR_403 | ENSG00000103197 | 16 | 2,047,465 | 2,088,720 |
| CNGB1 | ICR_445 | ENSG00000070729 | 16 | 57,882,340 | 57,971,116 |
| INPP5K | ICR_462 | ENSG00000132376 | 17 | 1,494,571 | 1,516,888 |
| PAFAH1B1 | ICR_465 | ENSG00000007168 | 17 | 2,593,210 | 2,685,615 |
| SLC25A39 | ICR_495 | ENSG00000013306 | 17 | 44,319,625 | 44324,870 |
| C3 | ICR_561 | ENSG00000125730 | 19 | 6,677,704 | 6,730,562 |
| MAST1 | ICR_568 | ENSG00000105613 | 19 | 12,833,951 | 12,874,951 |
| PGLYRP2 | ICR_573 | ENSG00000161031 | 19 | 15,468,645 | 15,498,956 |
| PLCB1 | ICR_714 | ENSG00000182621 | 20 | 8,077,251 | 8,968,360 |
| L3MBTL1 | ICR_755 | ENSG00000185513 | 20 | 43,507,680 | 43,550,950 |
| ZNF335 | ICR_759 | ENSG00000198026 | 20 | 45,948,653 | 45,972,172 |
| GNAS | ICR_766 | ENSG00000087460 | 20 | 58,839,718 | 58,911,192 |
| GNAS | ICR_767 | ENSG00000087460 | 20 | 58,839,718 | 58,911,192 |
| GNAS | ICR_768 | ENSG00000087460 | 20 | 58,839,718 | 58,911,192 |
| GNAS | ICR_769 | ENSG00000087460 | 20 | 58,839,718 | 58,911,192 |
| KCNQ2 | ICR_777 | ENSG00000075043 | 20 | 63,400,210 | 63,472,677 |
| KCNQ2 | ICR_778 | ENSG00000075043 | 20 | 63,400,210 | 63,472,677 |
| EEF1A2 | ICR_778 | ENSG00000101210 | 20 | 63,488,013 | 63,499,315 |
| MICAL3 | ICR_914 | ENSG00000243156 | 22 | 17,787,649 | 18,024,559 |
| TBX1 | ICR_918 | ENSG00000184058 | 22 | 19,756,703 | 19,783,593 |
| TBX1 | ICR_917 | ENSG00000184058 | 22 | 19,756,703 | 19,783,593 |
| MAPK1 | ICR_920 | ENSG00000100030 | 22 | 21,754,500 | 21,867,680 |
| UPB1 | ICR_923 | ENSG00000100024 | 22 | 24,494,107 | 24,528,390 |
| TNRC6B | ICR_935 | ENSG00000100354 | 22 | 40,044,817 | 40,335,808 |
| MAP3K15 | ICR_1404 | ENSG00000180815 | X | 19,360,056 | 19,515,261 |
| CNKSR2 | ICR_1406 | ENSG00000149970 | X | 21,374,418 | 21,654,695 |
| USP9X | ICR_1415 | ENSG00000124486 | X | 41,085,635 | 41,236,579 |
| ELK1 | ICR_1418 | ENSG00000126767 | X | 47,635,521 | 47,650,604 |
| HDAC6 | ICR_1419 | ENSG00000094631 | X | 48,801,377 | 48,824,982 |
| CACNA1F | ICR_1423 | ENSG00000102001 | X | 49,205,063 | 49,233,371 |
| TAF1 | ICR_1436 | ENSG00000147133 | X | 71,366,239 | 71,532,374 |
| KIAA1210 | ICR_1456 | ENSG00000250423 | X | 119,078,635 | 119,150,579 |
| IDS | ICR_1473 | ENSG00000010404 | X | 149,476,990 | 149,521,096 |
| MECP2 | ICR_1482 | ENSG00000169057 | X | 154,021,573 | 154,137,103 |
| MECP2 | ICR_1481 | ENSG00000169057 | X | 154,021,573 | 154,137,103 |
| RPL10 | ICR_1483 | ENSG00000147403 | X | 154,389,955 | 154,409,168 |
| RPL10 | ICR_1484 | ENSG00000147403 | X | 154,389,955 | 154,409,168 |
| RPL10 | ICR_1485 | ENSG00000147403 | X | 154,389,955 | 154,409,168 |

^{a}The ICR number listed is also the SEQ ID NO: in the Sequence Listing. For example, “ICR_5” corresponds to SEQ ID NO: 5, “ICR_373” corresponds to SEQ ID NO: 373, etc.
^{b}The start and end positions correspond to the nucleotide number in the corresponding chromosomal sequences of the Homo sapiens GRCh38.p13 Primary Assembly as set forth in the GENBANK ® biosequence database.

Another medical condition that is associated with the expression of particular genes, some of which have been shown to be associated by monoallelic expression, is schizophrenia. Genes that are associated with schizophrenia and are located within 10 kb of one or more of ICRs 1-1611 include but are not limited to those set forth in Table 3.

TABLE 3
Exemplary Genetic Loci Associated with Schizophrenia
and that are Within 10 kb of an ICR

| | | Ensembl Gene | Human | | |
|---|---|---|---|---|---|
| Symbol | ICR No.^{a} | Accession No. | Chromosome | Start^{b} | End^{b} |
| CAMTA1 | ICR_16 | ENSG00000171735 | 1 | 6,785,324 | 7,769,706 |
| TDRD5 | ICR_176 | ENSG00000162782 | 1 | 179,591,613 | 179,691,272 |
| SEMA3F | ICR_206 | ENSG00000001617 | 3 | 50,155,045 | 50,189,075 |
| ADRA2C | ICR_246 | ENSG00000184160 | 4 | 3,766,348 | 3,768,526 |
| KIF25 | ICR_417 | ENSG00000125337 | 6 | 167,996,241 | 168,045,089 |
| PLXNA4 | ICR_482 | ENSG00000221866 | 7 | 132,123,332 | 132,648,688 |
| PTK2B | ICR_517 | ENSG00000120899 | 8 | 2,7311,482 | 27,459,391 |
| TRAPPC9 | ICR_548 | ENSG00000167632 | 8 | 139,730,343 | 140,458,579 |
| TRAPPC9 | ICR_549 | ENSG00000167632 | 8 | 139,730,343 | 140,458,579 |
| FAM157B | ICR_629 | ENSG00000233013 | 9 | 138,216,187 | 138,252,994 |
| PDXDC1 | ICR_930 | ENSG00000179889 | 16 | 14,974,591 | 15,139,339 |
| ABCC3 | ICR_1020 | ENSG00000108846 | 17 | 50,634,777 | 50,692,252 |
| SEPT9 | ICR_1026 | ENSG00000184640 | 17 | 77,280,569 | 77,500,596 |
| MKNK2 | ICR_1063 | ENSG00000099875 | 19 | 2,037,465 | 2,051,244 |
| AP3D1 | ICR_1064 | ENSG00000065000 | 19 | 2,100,988 | 2,164,465 |
| SIGLEC9 | ICR_1133 | ENSG00000129450 | 19 | 51,124,908 | 51,136,651 |
| NHS | ICR_1400 | ENSG00000188158 | X | 17,375,420 | 17,735,994 |
| IGSF1 | ICR_1466 | ENSG00000147255 | X | 131,273,506 | 131,578,899 |
| MECP2 | ICR_1480 | ENSG00000169057 | X | 154,021,573 | 154,137,103 |
| MECP2 | ICR_1481 | ENSG00000169057 | X | 154,021,573 | 154,137,103 |

^{a}The ICR number listed is also the SEQ ID NO: in the Sequence Listing. For example, “ICR_16” corresponds to SEQ ID NO: 16, “ICR_206” corresponds to SEQ ID NO: 206, etc.
^{b}The start and end positions correspond to the nucleotide number in the corresponding chromosomal sequences of the Homo sapiens GRCh38.p13 Primary Assembly as set forth in the GENBANK ® biosequence database.

By way of further example and not limitation, the field of hepatology is in an intense search for biomarkers for liver cancer diagnostics and prediction in circulation (so called liquid biopsies) because unlike other cancers, tissue specimens are not available, as the vast majority are diagnosed by radiographic imaging. Hypermethylation of the ICR at the DLK1/MEG3 imprinted domain occurs frequently enough that it is widely proposed as a biomarker for a poorer prognosis among liver cancer cases (presumably via inactivation of the tumor suppressor MEG3 and other genes in this gene cluster). A persistent relationship between hypermethylation of the MEG3 sequence region and cadmium exposure has been reported, which is highly suspected to contribute to liver cancers in general, and may drive some of the increase in the incidence of hepatocellular carcinoma, specifically. In untargeted analyses based on whole genome bisulfite sequencing of liver tumors and adjacent tissues, we report that of the differentially methylated regions found between tumor and normal liver tissue, 548 overlap candidate ICRs, and 146 of those overlap annotated transcripts. In relation to autism, 1,904 associated genes have been reported (1,875 from the ENSEMBL hg38 version; see also Li et al., 2020). We found that 82 of the autisms-related genes are within 10kb of the ICRs. The list of the 82 genes is attached.

In some embodiments, a pattern of methylation of ICRs that is associated with a disease such as autism, early onset liver cancer or other subtype, e.g., hepatocellular- or cholangio-carcinoma, can be developed into a panel. Such a panel of methylation patterns can then be multiplexed and used to detect the presence/absence of disease, or prognosis, for example, if a certain threshold of the number of differentially methylated regions is reached. Similar sequencing technologies have been developed for cervical intra-epithelial neoplasia, where the presence or absence oncogenic human papilloma virus (HPV) viral DNA sequences is used to triage patients for further work-up. Once the biological sample is isolated from a subject and used to interrogate the array, the data that is returned can be compared to one or more profiles previously established for diseases (which in some embodiments can be a continually expanding process), with prognosis for disease susceptibility based on matching known profiles.

In some embodiments, a DNA methylation status comprises one or more epigenomic features of at least one imprinted gene. By way of example and not limitation, the one or more epigenomic features can comprise a methylation profile of the subject with respect to at least one imprinted gene. Also, by way of example and not limitation, the one or more epigenomic features are selected from the group consisting of a DNA sequence methylation state, a nucleosome positioning feature, and a histone modification.

Genes for which expression is associated with medical conditions can include genes that are aberrantly upregulated or downregulated, with the aberrant upregulation or downregulation occurring in a temporal, cell type-specific, organ type-specific, and/or tissue-specific manner. By way of example and not limitation, it is known that monoallelic gene expression is particularly relevant to proper development of mammals. In some embodiments, this monoallelic gene expression persists from very early development (e.g., is already specified in the one cell fertilized embryo or soon thereafter during embryogenesis).

It is also known that modifications of DNA methylation can change as a subject ages, and in some embodiments these changes either result in or can be enhanced by disease processes. Specific DNA methylation differences can thus include one or more epigenomic features that relates to a gene for which expression or lack of expression is associated with the medical condition. The identification of ICRs 1-1611 as set forth herein has thus enhanced the ability to correlate express or lack of expression of imprinted gene with particular medical conditions.

In some embodiments, the medical condition is Alzheimer's disease (AD). Using AD solely as a representative medical condition, in some embodiments the presently disclosed subject matter relates to methods for detecting a presence of and/or a susceptibility to AD. In some embodiments, at least one imprinted gene is thus a gene that is associated with Alzheimer's disease. In some embodiments, genes that are in proximity to a genetic locus that is associated with certain epigenomic features, correlated expression in association to epigenomic features, or reported association to Alzheimer disease in combination with either of the first two criteria can be identified that relate to AD development and/or progression. Particularly where changes in imprinting status of one or more genes and/or one or more ICRs associated with those one or more genes that themselves are associated with AD development and/or progression can be detected, said changes in imprinting status can be employed to detect a presence of and/or a susceptibility to AD. A kinase anchor protein 2 (AKAP) is shown as an example of ICRs region that overlap with DMR identified in AD patient. This gene was associated with AD disease in GWAS studies (Poelmans et al., 2013). The regulatory mechanism behind this association is not fully known. In some embodiments, an imprinting status of one or more of ICRs 1-1611 and/or the genomic regions associated with SEQ ID NOs: 1612-1816 is determined for a subject and compared to an imprinting status of the same one or more of ICRs of an appropriate control group, which in some embodiments can be a control group that has been shown to not develop AD.

In some embodiments, in view of the fact that imprinting status can be specified very early in development, it is even possible to detect a presence of and/or a susceptibility to a medical condition associated with monoallelic expression in a subject at a time that is long before any symptoms develop in the subject. In this regard, the compositions and methods of the presently disclosed subject matter provide a significant advantage over RNA-based gene expression analysis in that imprinting statuses can be set long before any relevant gene expression must occur. For example, a susceptibility to a late onset medical condition can be detected decades before the medical condition manifests itself in a subject because the imprinting status that is associated with the medical condition exists when the subject is born (in fact, earlier).

Another advantage of the presently disclosed compositions and methods is that they rely on interrogatable characteristics of subjects that are generally not cell-type, tissue-type, or organ-type specific, and thus any biological sample that can be isolated from a subject can be assayed. Typically, differences in gene expression must be assayed at a time and in a cell, tissue, and/or organ where the gene expression differences take place. It is not the case that such a cell, tissue, or organ can always be biopsied (e.g., for neurological diseases), nor is it generally preferable to have to wait for an onset of symptoms to perform the gene expression analysis even in accessible cells, organs, or tissues as the changes in gene expression might be causative of the medical condition. The presently disclosed compositions and methods provide for analysis of any cell, tissue, or organ, and including cells, tissues, and organs that are unaffected and/or will be unaffected by the medical condition, such as but not limited to a blood sample, that can be isolated at any stage of development (e.g., from a newborn, a young child, and/or from an adult). Thus, in some embodiments the presently disclosed compositions and methods provide for diagnosis of medical conditions at much earlier stages (including but not limited to times longer before a medical condition occurs or worsens) using biological samples that themselves need not be affected by the medical condition.

As such, in some embodiments the presently disclosed subject matter relates to methods for predicting a susceptibility to future development of a medical condition associated with monoallelic expression in a subject prior to the onset of any symptoms of the medical condition in the subject. In some embodiments, the methods comprise, consist essentially of, or consist of (a) obtaining a biological sample from the subject, wherein the biological sample comprises one or more nucleic acid molecules that correspond to an Imprint Control Region (ICR) selected from the group consisting of ICRs 1-1611 or a subset thereof; (b) analyzing the one or more nucleic acid molecules to determine the DNA methylation status of one or both alleles of at least one imprinted gene associated with at least one of ICRs 1-1611; and (c) determining whether the DNA methylation status determined correlates with future development of the medical condition, whereby a susceptibility to future development of the medical condition is predicted. In some embodiments, the biological sample comprises genomic DNA isolated from any cell, tissue, or organ of the subject, including a cell, tissue, or organ that is generally unaffected in subjects who have the medical condition, and not necessarily usable for diagnosis of conditions affecting target tissues by means specific to those affected tissues, including, but not limited to, physical morphology, immunological assays, protein expression, or RNA expression.

III.C. Methods for Monitoring the Progression of Medical Conditions Associated with Monoallelic Gene Expression in Subjects

It is noted that not all medical conditions associated with monoallelic expression are caused by static imprinting statuses specified early in development. Certain medical conditions arise form undesirable changes in the imprinting status of one or more genes associated with the medical condition, which in some embodiments can be reflected in changes in the methylation of one or more of ICRs 1-1611. In such a case, it is possible to employ the compositions and methods of the presently disclosed subject matter to monitor progression of a medical condition by detecting changes that occur in the methylation of one or more of ICRs 1-1611 in a subject over time.

Thus, in some embodiments the presently disclosed subject matter relates to methods for monitoring the progression of a medical condition associated with monoallelic expression in a subject, wherein the methods comprise, consist essentially of, or consist of (a) obtaining a biological sample from the subject, wherein the biological sample comprises one or more nucleic acid molecules that correspond to an Imprint Control Region (ICR) selected from the group consisting of ICRs 1-1611 or a subset thereof; (b) analyzing the one or more nucleic acid molecules to determine the DNA methylation status of one or both alleles of at least one imprinted gene associated with at least one of ICRs 1-1611; (c) identifying one or more changes that have occurred in the DNA methylation status of the ICRs analyzed in the subject; and (d) determining of the one or more changes identified correlate with a progression of or an improvement in at least one symptom of the medical condition, wherein the determining step provides monitoring of the progression of the medical condition associated with monoallelic expression in the subject. In some embodiments, the identifying comprises comparing a first methylation status with respect to the at least one of ICRs 1-1611 in the subject to a second, subsequent methylation status with respect to the at least one of ICRs 1-1611 in the subject, wherein the comparing provides an indication of the second, subsequent methylation becoming more or less similar to the methylation status with respect to the at least one of ICRs 1-1611 in normal subjects.

III.D. Methods for Monitoring Treatments of Medical Conditions Associated with Monoallelic Gene Expression in Subjects

Similarly, in some embodiments the presently disclosed subject matter relates to methods for monitoring treatments for medical conditions associated with monoallelic expression in subjects. In some embodiments, the methods comprise, consist essentially of, or consist of (a) obtaining a biological sample from the subject, wherein the biological sample comprises one or more nucleic acid molecules that correspond to an Imprint Control Region (ICR) selected from the group consisting of ICRs 1-1611 or a subset thereof; (b) analyzing the one or more nucleic acid molecules to determine the DNA methylation status of one or both alleles of at least one imprinted gene associated with at least one of ICRs 1-1611; (c) identifying one or more changes that have occurred in the DNA methylation status of the ICRs analyzed in the subject subsequent to treatment versus prior to treatment or prior to a specific treatment occurrence; and (d) determining of the one or more changes identified correlate with a progression of or an improvement in at least one symptom of the medical condition, wherein the determining step provides monitoring of the effectiveness or lack thereof of the treatment of the medical condition associated with monoallelic expression in the subject. In some embodiments, the identifying comprises comparing a first methylation status with respect to the at least one of ICRs 1-1611 in the subject to a second, subsequent methylation status with respect to the at least one of ICRs 1-1611 in the subject, wherein the comparing provides an indication of the second, subsequent methylation becoming more or less similar to the methylation status with respect to the at least one of ICRs 1-1611 in normal subjects. In some embodiments, the first methylation status is determined prior to the initiation of any treatment, prior to the initiation of a new treatment, and/or prior to the administration of a subsequent treatment. In some embodiments, the second methylation status is determined after the initiation of the first treatment or any subsequent treatment.

Thus, in some embodiments the second methylation status and the first methylation status relate to a subject that has undergone no additional treatment since the first methylation status was determined, and the second methylation status reflects only the passage of time during which the first treatment has been acting. However, in some embodiments at least one difference in treatment between the first and second methylation status determinations, whether that one difference is a new treatment, a subsequent administration of the same treatment, and/or a change in the nature of the treatment (e.g., a modification of dose, administration frequency, and/or route of administration, etc.).

In some embodiments, the treatment being monitored is a standard treatment designed to modify one or more symptoms of the medical condition and thus is not designed to directly modify the methylation status of any genetic locus associated with monoallelic expression in a subject. However, in some embodiments the treatment being monitored is designed to directly modulate the methylation status of at least one genetic locus associated with the medical condition. By way of example and not limitation, in some embodiments the treatment is designed to directly reverse some undesirable methylation difference that has occurred in a gene associated with a medical condition, wherein the deleterious methylation difference gives rise to and/or exacerbates at least one symptom, characteristic, or feature of the medical condition.

Stated another way, in those embodiments where a difference in methylation status of a genetic locus associated with a medical condition (e.g., one or more of ICRs 1-1611) bears a causal relationship to at least one symptom, characteristic, or feature of the medical condition, a treatment can be devised to “reverse” or “normalize” the methylation status of a genetic locus in order to decrease or eliminate the consequence of the deleterious methylation difference. The compositions and methods of the presently disclosed subject matter can be employed to detect these deleterious differences and monitor any changes that occur in the methylation statuses in relevant subjects, such as but not limited to changes that occur in the methylation statuses of one or more of ICRs 1-1611 in subjects.

III.E. Methods for Preventing Development of and/or Treating Medical Conditions Associated with Monoallelic Gene Expression in Subjects

Accordingly, in some embodiments the presently disclosed subject matter also relates to methods for preventing development of and/or treating medical conditions associated with undesirable changes in methylation statuses of genetic loci associated with monoallelic gene expression in subjects by directly modifying genomic DNA, particularly with respect to methylation statuses. In this regard, when changes in methylation status of relevant genetic loci occur or are specified in a particular individual, altering those methylation statuses by direct modification of genomic DNA should prevent development of the medical condition and/or ameliorate at last one symptom, characteristic, or feature of the medical condition. By way of example and not limitation, direct genomic DNA modifications can be induced using the CRISPR/Cas system as described, for example, in U.S. Pat. Nos. 8,697,359 and 9,688,971, both of which are incorporated by reference herein in their entireties.

### EXAMPLES

The following EXAMPLES as set forth herein have been presented for purposes of illustration and description. These EXAMPLES are not intended to limit the disclosure to the form disclosed herein, as variations and modifications commensurate with the teachings of the description of the disclosure, and the skill or knowledge of the relevant art, are within the scope as set forth herein. It is intended that the appended claims be construed to include alternative embodiments to the extent permitted by the prior art.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Introduction to the Examples

Data accumulated over the last two decades support the fetal origins of adult disease susceptibility hypothesis, and increasingly, the mediating role of epigenetic mechanisms. This implies that implicated epigenetic marks can be developed into screening tools to predict future risk of disease. However, epigenetic marks identified in accessible tissues like peripheral blood do not always correlate with those of inaccessible tissues relevant to the disease under study, and they may also be epiphenomena, i.e., being altered by disease.

Data were collected to characterize parent-of-origin differential methylation consistent with imprint control regions (ICRs) that regulate monoalleleic gene expression of developmentally significant genes; and to identify ICRs associated with Alzheimer's disease (AD) in African Americans and European Americans.

Whole genome bisulfite sequencing (WGBS) and pyrosequencing were employed to sequence DNA derived from the endodermal, mesodermal, and ectodermal germ layers, the gametes and accessible CD14—and human umbilical cord vein endothelial cells and developed a pipeline to identify putative ICRs. The overlap of ICRs with differentially methylated regions (DMRs) from frontal cortex-derived DNA from Alzheimer's disease (AD) cases and controls was then assessed.

From DNA obtained from multiple ethnic groups, 1,495 human ICRs were identified, including the 24 already characterized, and validated a subset in multiple accessible and inaccessible tissues. The average ICR contains 23 CG dinucleotides, and ranges from 13 to ˜4,000 bp, in general, larger than previously characterized ICRs. In frontal cortex-derived DNA from cases and controls, 31,600 differentially methylated regions (DMRs) were associated with AD in AAs (p<1.70×10⁻⁶¹), and 740 associated in EAs (p<1.50×10⁻⁹⁰, a more than 40-fold difference, with 89 regions found in both. Additionally, 8% (119/1,495) of AD-associated DMRs coincided with ICRs, some previously implicated in AD.

Disclosed herein is a map of human ICRs that should accelerate the discovery of early acquired, disease-related differential methylation. The significant representation of candidate ICRs (8%) in AD-associated DMRs support the utility of these regions in early detection of a devastating disease for which pharmaceutical interventions may delay progression. Large-scale population-based studies are required to refine diagnostic targets.

### Materials and Methods for the Examples

Participants. Tissues from 12 conceptuses aged 65-95 days, with no apparent developmental abnormalities, of both sexes (confirmed by sex linked marker genotyping); both AAs and EAs were selected. These tissues were obtained from the National Institutes of Health funded Laboratory of Human Embryology at the University of Washington, Seattle, Wash., and snap frozen to preserve DNA/RNA integrity (NCSU Institutional Review Board #3565). Conceptus tissues are ideal for identifying ICRs because the gametic and somatic imprint marks are intact, and monoallelic gene expression of imprinted genes occurs primarily during embryonic development (Lambertini et al., 2012; Ishida & Moore, 2013; Green et al., 2015).

Whole genome bisulfite sequencing. Libraries for NextSeq sequencing were prepared from bisulfite converted DNA, with 27 of 36 samples passing quality control standards for sequencing by Illumina NextSeq with 12-15× sequencing coverage. Libraries were index-tagged for separating reads after multiplex sequencing, pooled into groups of nine, with each group split for sequencing on three separate lanes. Splitting samples across lanes ensured that no single sample is disproportionally affected by technical variability specific to an individual sequencing lane such as low read numbers or low read quality. For quality control, sample specific problems related to library quality were identified by consistent low quality of specific samples across lanes and affected samples were then re-run or removed from analysis if the problem persisted.

Bioinformatic approaches to identify ICRs. Samples were separated by index sequences, and aligned to a reference in silico bisulfite converted genome, eliminating reads without unique alignment to the reference sequence (due to either repetitive sequence or loss of specificity from bisulfite conversion of cytosines), and duplicate reads (indicative of clonal amplification of original random DNA fragments). From these reads, methylation fractions and read counts were calculated for all CpG sites in the genome.

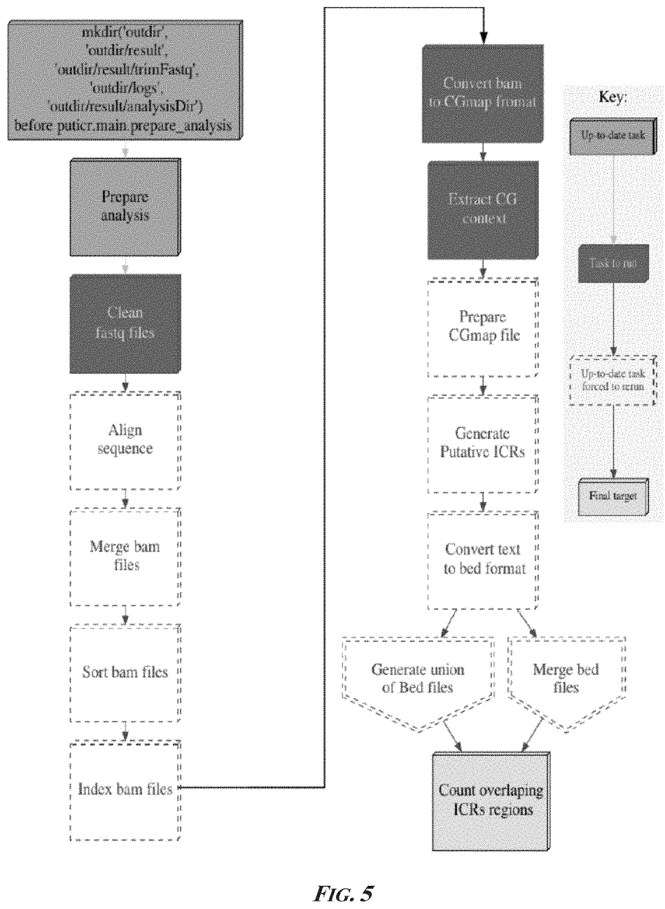

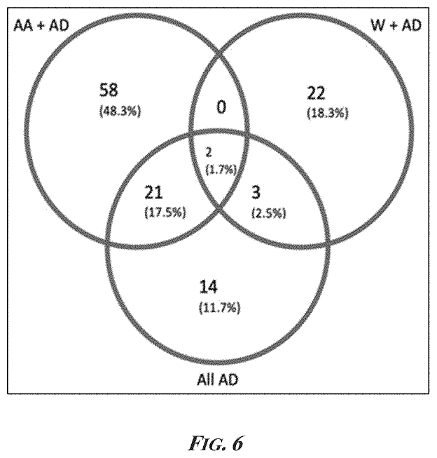

We developed a puticr application using a Ruffus framework in Python. The workflow is detailed in FIG. 5. This application is designed to scan the genome and identify regions of allelic differential methylation based on some or all of the following four criteria: 1) ≥5 consecutive CpG sites, consistent with a cis-acting regulatory sequence, 2) methylation levels of ˜50%+/−15% at each site (i.e., 35%-65% methylation), consistent with monoallelic methylation (100% in one parental allele and 0% on the other), 3) similarity of methylation levels across the three germ layers, consistent with methylation being established before gastrulation, thus similar in all tissues, and 4) similarity of methylation across individuals, consistent with regulation of critical processes in early development, that do not vary by sex, ethnicity, developmental age, or person-to-person. In addition, fully methylated or unmethylated regions from oocyte and sperm sequences were also compared, as these are the original parent-of-origin specific regions.

Identification of aberrantly methylated ICRs in a complex human disease. To determine the role of ICR methylation in early detection of Alzheimer's disease differential methylation of ICRs, we obtained from the Duke University Bryan Brain Bank frontal cortex-derived DNA from 8 AAs and 8 EAs (4 in each ethnic group with Alzheimer's disease and 4 controls). We used whole genome bisulfite sequencing with 30× coverage, and bioinformatically identified regions of differential methylation between cases and controls overall and within each ethnic group.

### Example 1

### Characteristics of Human Imprint Control Regions (ICRs)

From the 36 libraries prepared from kidney, liver, and brain of 12 individuals (6 male, 6 female), 27 passed quality checks for Illumina NextSeq. The average number of reads for the 27 samples was 153 million (range 74-231 million), covering an average of 23.1 billion bases per sample (range 11.2-34.9 billion). Approximately 80% of reads had unique alignment to an in silico bisulfite converted human genome, and of 29.2 million CpG sites in the human genome, an average of 26.6 million (91%, range 86-94%) were covered by aligned reads for the set of 27 samples.

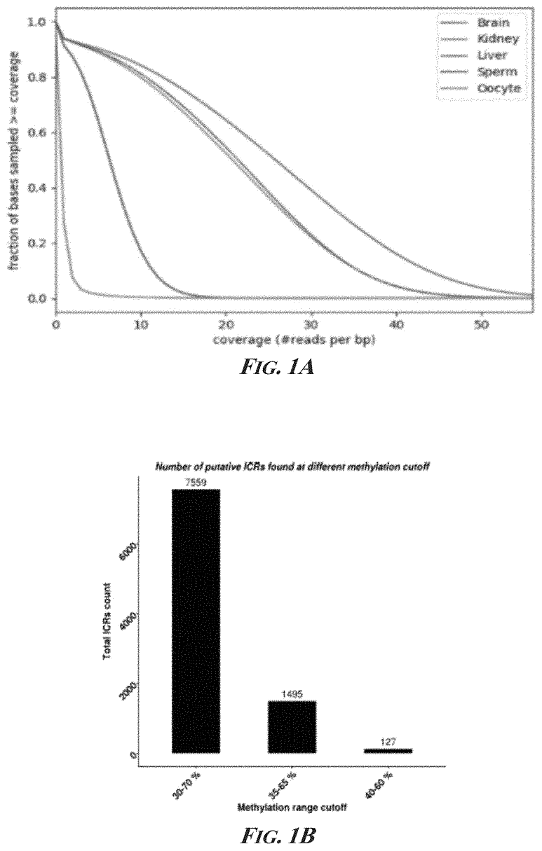

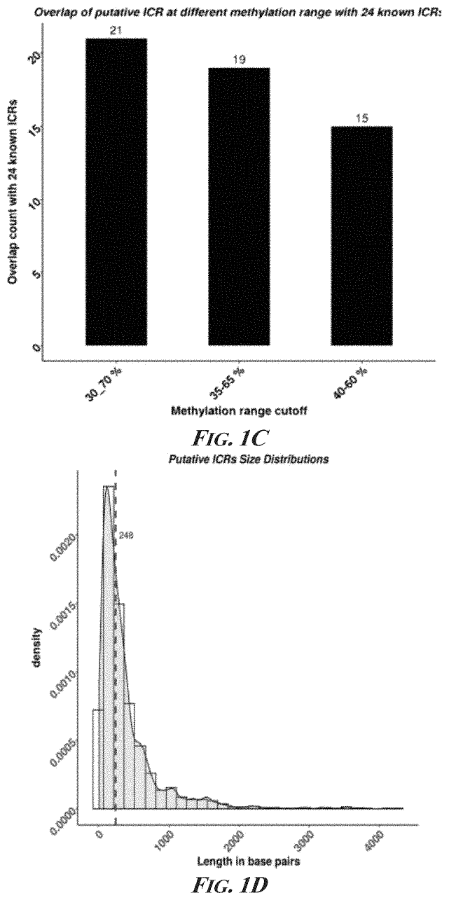

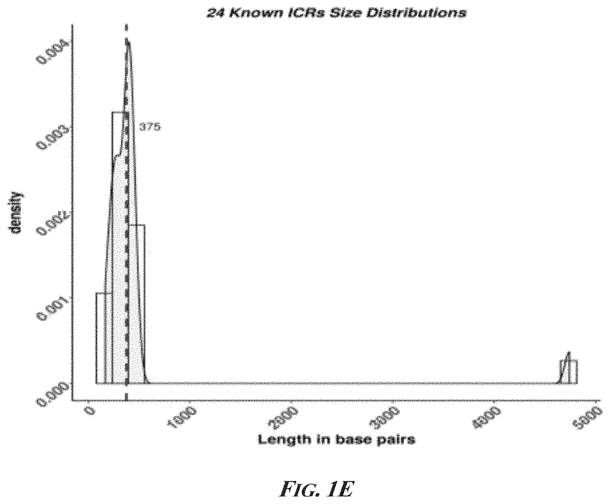

More than 75% of sequences obtained from the liver, brain and kidney DNA have sequence coverage >20× while the oocyte and sperm have a lower coverage as compared to those obtained for the three somatic tissues (FIG. 1A). Using methylation percentages calculated for CpG sites, candidate ICRs were defined based on the criteria of a 300 bp region with five or more consecutive CpG sites with individual methylation level of approximately 50% ±15% (35% to 65%) in somatic tissues; this is consistent with one parental allele being fully methylated while the other is unmethylated acting in cis for >80% of the sites. These regions of differential methylation also had to align in DNA derived from tissues representing the three germ layers, consistent with the establishment of these methylation marks before gastrulation, and the requirement that an ICR be in all cell types. Consistent with their function to control gene dosage in all individuals, these methylation marks also had to be similar across individuals. Using the most relaxed criteria 50%±20% (30% to 70%), we identified 7,559 putative ICRs, including most (88%) of known ICRs (FIG. 1B). FIGS. 1B and 1C also shows that tightening this methylation fraction window to 50% ±15% (35% to 65%) resulted in a decrease in the number of putative ICRs to 1,495 putative ICRs, including ˜80% of known ICRs. Further restricting the window to 50%±10% (40% to 60%) decreased the number of candidate ICRs to 127, including 63% of known ICRs.

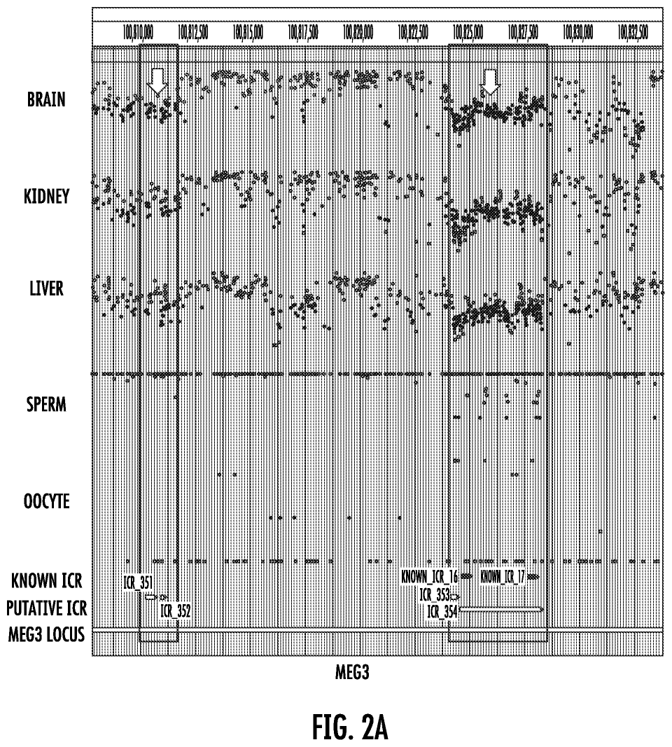

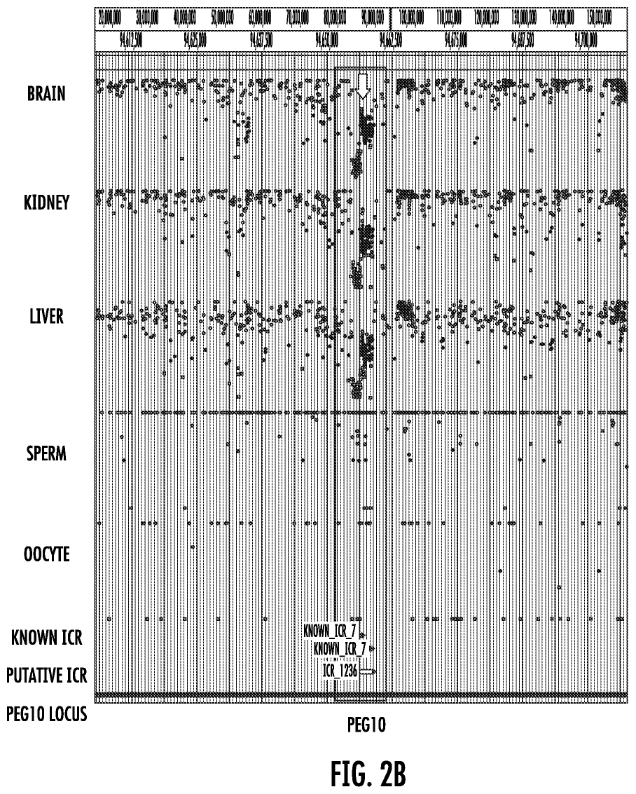

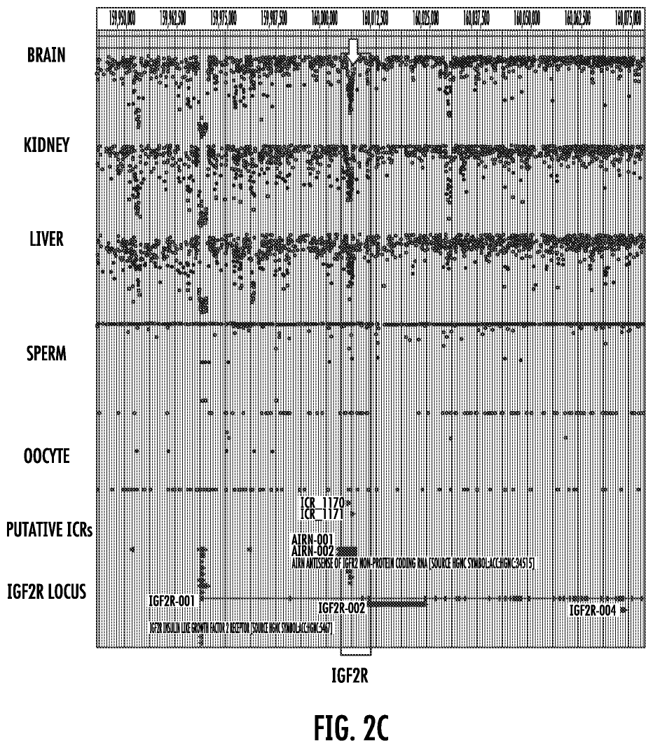

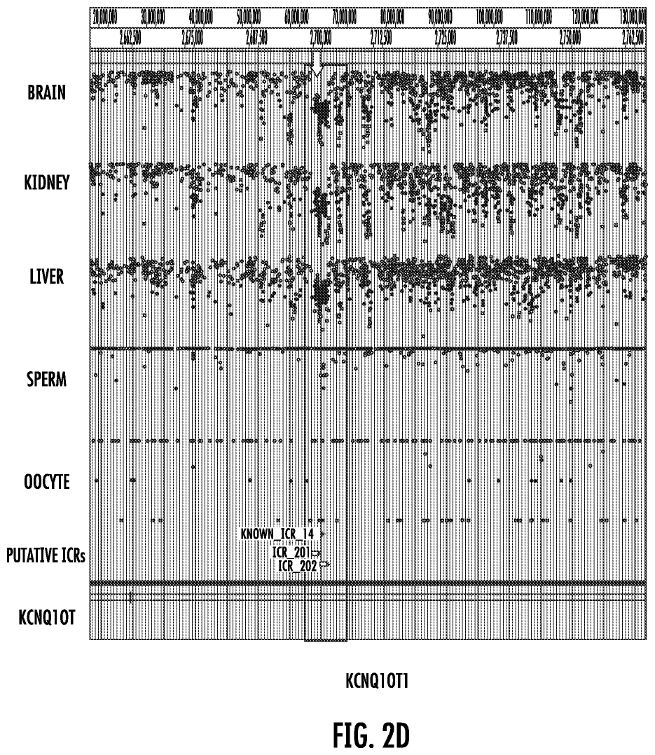

Public databases for oocyte sequences (Accession No. JGAS00000000006, accessible through the website of the Japanese Genotype-phenotype Archive; see also Okae et al., 2014; incorporated herein by reference in its entirety) were used to identify regions of parent-of-origin reciprocal gametic methylation, e.g. 100% methylation in sperm, and 0% in ooctyes, or vice-versa, among those meeting the initial ICR criteria. This pattern is indicative of gametic differential methylation, which can persist through fertilization and development, and propagate into somatic differential methylation in the offspring. We superimposed DNA sequences for the 1,495 candidate ICRs on sequence data from oocytes and sperm that were either unmethylated, i.e., 0-10%, or fully methylated, i.e., 90-100%. The length of known ICRs are comparable to those of novel ICRs, ranging in size from 13-4,000 bp with a median of 375 bp for the known 24 ICRs (FIG. 1D), and median 248 bp for the 1,476 novel ICRs (FIG. 1E). Based on the methylation profiles of 27 individuals in the kidney, brain, liver and the gametes, FIGS. 2A-2D depict screenshots of the application (putICR) for the previously described MEG3, PEG10, and KCNQ1OT1 locus (Chr11:2,685,000-2,700,000) and previously unknown IGF2R. FIGS. 2A-2D also show DNA methylation levels in the three tissues around the expected ˜50% level, along with coinciding reciprocal gametic methylation, defining a region 10 times longer than is currently defined for this ICR. The 1,495 ICR sequences are found in the Sequence Listing.

### Example 2

### Similarity of Methylation of Marks Across Accessible Tissues

For epidemiologic inquiry, a critically important quality of DNA methylation marks is that they are replicable regardless of sequencing technologies, and are similar across tissues, such that accessible tissues in otherwise healthy humans, who often serve as controls, can serve as surrogates for inaccessible target tissues. Such cell types would be those found in peripheral blood or maternal or fetal tissues discarded at birth, such as decidua, the fetal side of the placenta, or human umbilical vein endothelial cells (HUVECS) present in the umbilical cord. To directly address this issue, we randomly selected a set of known and novel/putative ICRs to 1) replicate the findings that these methylation marks are ˜50% in the three germ layers, that 2) the 50% methylation was similar in DNA derived from multiple accessible tissues, and that 3) these methylation marks were similar in an independent sample of individuals.

For confirmation by a second sequencing method, pyrosequencing results from one of the novel ICRs, a sequence region in chromosome 2, comparing methylation measured by WGBS and pyrosequencing, which shows ˜50% methylation levels across multiple tissues. This and other regions were selected based on neighboring genes, correlated RNA expression, and/or somatic and gametic methylation that most closely fit the criteria. The similarity of methylation marks across tissues for the brains, kidneys, and livers of the nine individuals included in the WGBS data set, the data used to initially define the ICR, is shown in FIGS. 2A-2D.

To examine whether methylation marks at novel and known ICRs are similar across multiple tissues that are accessible in otherwise healthy humans, we used previously developed pyrosequencing assays (Murphy et al., 2012; which is incorporated herein by reference in its entirety) to measure methylation levels of the known ICRs regulating the imprinted expression of IGF2, PEG3 and PEG10 and juxtapose these with the novel ICRs proximal (and potentially regulatory) to GATA3 in Chr10, and RGPD1 in Chr 2. Methylation marks measured in adult mixed blood leukocytes, isolated CD14-monocytes from newborn cord blood, and HUVECs at the known ICRs regulating the imprinted expression of IGF2, PEG3 and PEG10. Methylation marks across the same tissues for the putative ICRs proximal to GATA3, and RGPD1 in Chr 2. For both the previously known and the putative ICRs, methylation is highly consistent across cell types and individuals, in the defining ˜50% range. Methylation marks in other tissues (including lung, gut, adrenal gland, spleen, thymus, and pancreas) and term placenta and fetal placenta also exhibit similar methylation marks.

### Example 3

### Functional Significance of ICR Methylation

To determine the extent to which putative ICRs are likely functional, we used a combination of public databases including the Analysis of Motif Enrichment (AME) application (accessible through the website of The MEME Suite) and the Comparative Toxicogenomics Database (CTD) to examine molecular functions and metabolic pathways associated with 1,495 putative ICRs. These analyses suggest that 914 genes are within 5,000 bp upstream and downstream of these putative ICRs, of which 17 were not recognizable by CTD. Of the remaining 897 genes, 374 genes were associated with “protein binding activity”, 70 genes were associated with “transcription regulator activity”, 52 genes were associated with “DNA-binding transcription factor activity,” 81 genes were associated with “DNA binding” and 29 genes were associated with “transcription co-regulator activity.” Approximately one third (n=253) of these 897 genes were related to “ion binding” including 185 genes “metal ion binding”, 186 genes “cation binding” and 59 genes “calcium ion binding.” The CTD enrichment pathway analysis also revealed nine pathways that were associated with these 897 genes close to 1,495 ICRs. These pathways were neuronal system and transmission across chemical synapses pathways, pathways in cancers, signal transduction, circadian entrainment, axon guidance, cholinergic synapse, glutamatergic synapse, and calcium signaling pathways. Remarkably, 17 genes were known to be associated with Alzheimer's disease.

### Example 4

### Association of ICRs with Major Human Diseases: Alzheimer's Disease as an Example

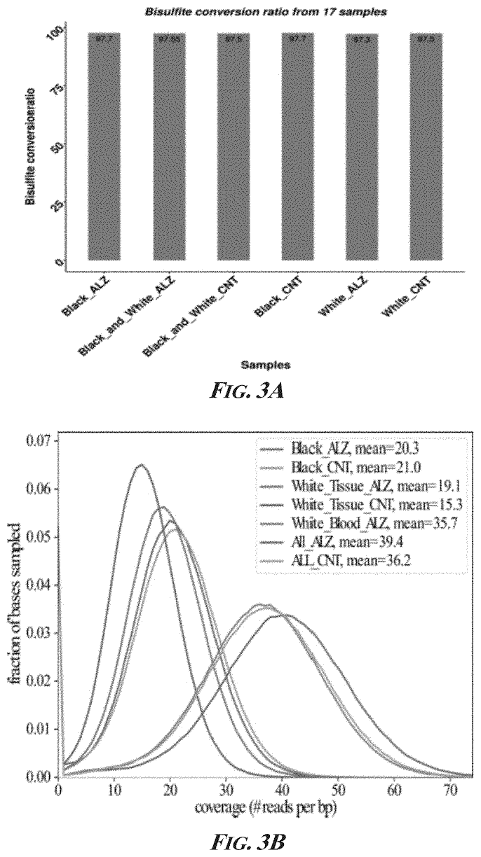

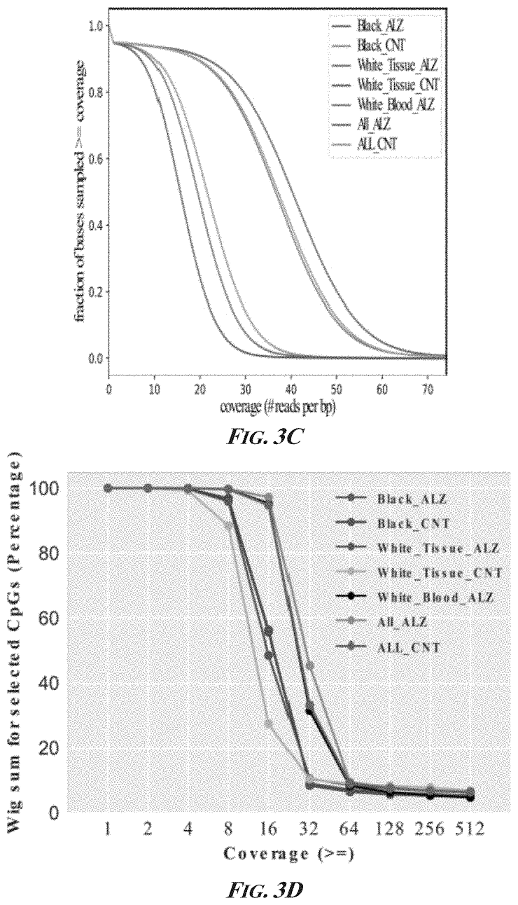

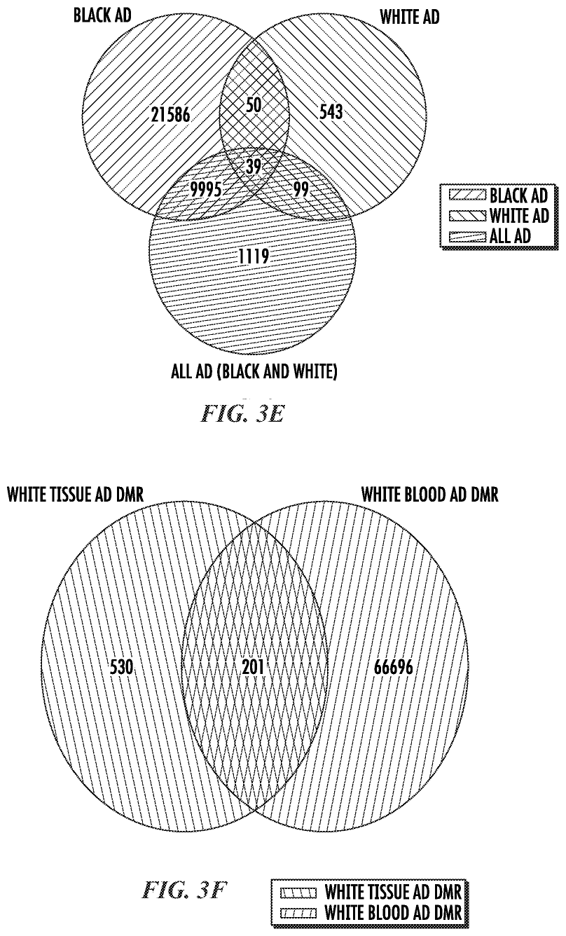

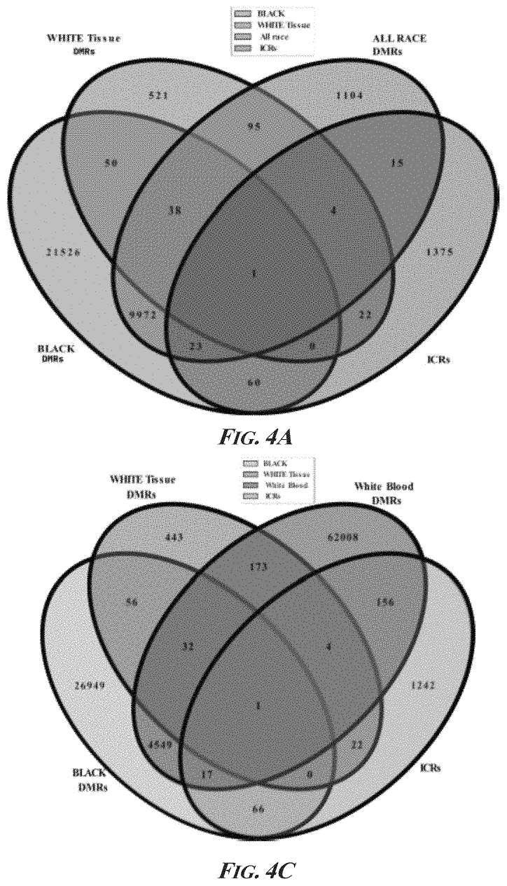

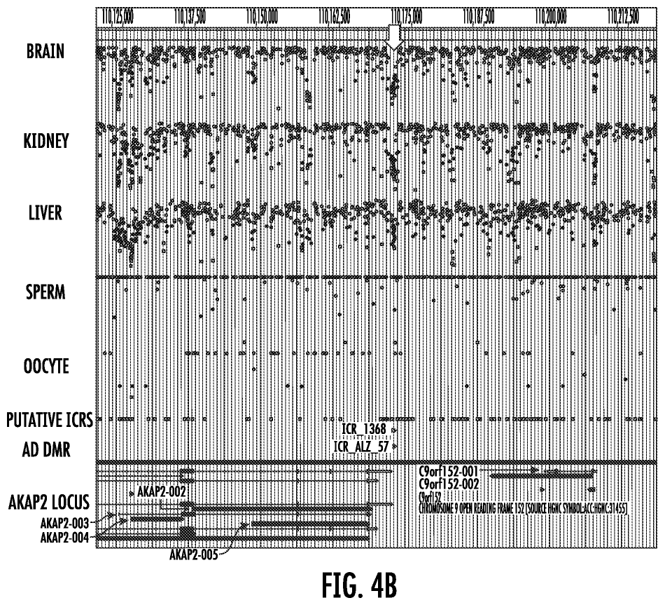

To determine whether there is a set of aberrantly methylated ICRs, likely established in development and stable throughout the life course, that are associated with AD risk, we used whole genome bisulfite sequencing to sequence DNA derived from the frontal cortex of eight AD males and females (four AAs and four EAs) and age, sex and ethnicity-matched control brains. FIG. 3A shows that using criteria that define differentially methylated regions (DMRs) as regions with at least four CpG sites within 300 bp, with absolute methylation changes of at least 10% and covered with at least seven sequence reads (Sun et al., 2014), in the same direction (all increased or all decreased). In all cases, the bisulfite conversion was greater than 97% (FIG. 3A), and the sequence coverage ranges between 15×-36× (FIGS. 3B and 3C) with no sequence duplication bias (FIG. 3D). We identified ˜31,600 DMRs in AA AD samples, 731 were identified in EAs, and 11,252 were found in AD samples when samples were combined regardless of ethnicity (FIG. 3E). Of these, 89 were common between AAs and EAs. The overall overlap between AAs, EAs and combined races DMRs, and ICRs are shown in FIG. 4A. Of these, 89 were found in both AAs and EAs. Interestingly, when we compared the AD-related DMRs to the set of candidate ICRs, 84 DMRs among AAs overlap 81 ICRs, while 27 DMRs from EAs overlap 27 ICRs. For DMRs identified in combined ethnicities, 52 overlap 40 DMRs. In total, 120 of the 1,495 candidate ICRs overlap DMRs defined in AD frontal cortex tissue.

FIG. 3B shows examples of the sequence regions near AKAP2, which are AD-related loci that also overlap with novel putative ICRs. Furthermore, consistent with APOE genetic or epigenetic variation associated with Alzheimer's disease being only sporadically found in AA individuals (19), the 31,600 DMRs associated in AAs do not include the APOE locus. Intriguingly, the ICR that overlaps with APOE is one of 89 DMRs that are AD-related in the 120 ICRs we found in AAs and in EAs.

Among EA donors for whom blood DNA was also available, and therefore sequenced, over 66,000 DMRs were identified between cases and controls. Of these 210 were in common with DMRs from the brain tissue of EAs, 168 overlapped with putative ICRs, and five DMRs are found in both blood and tissue, and overlapped an ICR. Of these 5 DMRs, one of these genes mapped to known ICRs, two mapped close to piRNA reported as a signature for AD (e.g., AKAP2 in FIG. 4) (20), and two proximal to known genes, one of them implicated to AD. The two in proximity to zinc-finger transcription factors, ZNF429 and ZNF597, are imprinted (maternally expressed) with deletion resulting in defects in neural development.

We performed CTD Batch Analysis of 82 genes within 5,000 bp upstream and downstream of 119 putative ICRs which are found differentially methylated in Alzheimer's Disease patients. From these 82 genes, five genes were associated with transcription co-activator activity (MAML2), DNA-binding transcription activator activity (NFATC1, NFIC), DNA-binding transcription factor activity (NFATC1, NFIC, PEG3), DNA-binding transcription repressor activity (NFATC1, ZNF536), RNA polymerase II transcription coactivator binding (NFATC1), RNA polymerase II transcription factor binding (NFATC1), and transcription factor binding (NFATC1). Furthermore, the Reactome software (accessible through the website of Reactome) for pathway analyses revealed that 31 out of 82 identifiers in the sample were found in Reactome, where 219 pathways were activated by at least one of them. The top five pathways determined were prostacyclin signaling through prostacyclin receptor (GNAS), PKA activation in glucagon signaling (GNAS), Glutamate Neurotransmitter Release Cycle (NFATC1, PPFIA4), ADORA2B mediated anti-inflammatory cytokines production (ADM2, GNAS, GPR45), and glucagon-type ligand receptors (GNAS). These in silico observations support that the majority of the 1,495 putative ICRs identified here are functional, and a number are involved in neurodevelopment and function.

### Example 5

### Identification of Differentially Methylated Regions (DMRs) Related to Alzheimer's Disease

To identify AD-related genomic locations including ICRs that are relevant for at least two racial/ethnic groups, DMRs were identified from brain tissue of Alzheimer's patients as compared to age matched controls, as consecutive CpG sites with altered methylation associated with the disease state. DMRs that showed highest changes in methylation level, and strongest consistency across individuals were selected. We performed differentially methylated region (DMR) analysis of temporal lobe derived brain genomic DNA from African Americans (5 AD cases, 4 Controls), and Whites (4 AD cases, 4 Controls). African Americans with AD had four times as many DMRs (˜31.6K) compared to Whites with AD (731); the significantly higher number of DMRs in Blacks with AD may well reflect the disproportionately higher accumulations of ‘insults’ throughout the life course, effects of social and health disparities.

Comparing cases and controls with ethnicities merged resulted in 11,252 DMRs associated with AD.

To identify DMRs likely acquired before gastrulation that are likely stable when occurring on ICRs and can be detected in peripheral tissues such as blood, we examined the overlap between AD temporal lobe derived brain DMRs and the ICRs 1-1495, observing a total of 120 ICRs overlapping with AD DMRs, 81 of which were found in Black AD cases compared to 27 in White AD cases (with only 2 in common), and 14 DMRs that were only significant in the merged datasets (FIG. 6).

When taken together, these data were consistent with stable epigenetic dysregulation acquired before gastrulation, and therefore detectable in all tissues/cell types, contributing to Alzheimer's Disease in African Americans and Whites, and therefore hold enormous prospects for early detection using accessible cell types.

### Discussion of the Examples

Thus far, investigations into the developmental origins of disease susceptibility have been stymied by our limited knowledge of where recognizable patterns of these effects can be detected and quantified in the epigenome, particularly when using DNA from sample types accessible in otherwise healthy human populations. While sequence regions controlling the monoallelic expression of imprinted genes have been previously proposed as targets for such studies (Hoyo et al., 2009), only 24 of these regions had been described (Skaar et al., 2012), with potentially hundreds more unknown.

A significant contribution with respect to the presently disclosed subject matter is the characterization of 1,611 putative human ICRs and validation using genomic DNA obtained from multiple ethnic groups and tissues and cell types. These novel ICRs have an average CpG dinucleotide content of 23, and size range 13 to ˜4,000 bp. They also have characteristics very similar to the 24 previously characterized ICRs, while in many cases, expanding the regions for the 24. Ninety percent of these putative ICRs are in genomic regions of functional importance to gene regulation, including 30% (n=453) in regions of DNASE1 hypersensitivity and 22% (n=328) overlapping transcription factor binding sites. Refining the boundaries of these ICRs will be iterative, nonetheless, given the broad interest and the known importance of ICRs in early development, WGBS data for tissue derivatives of the three germ layers and sperm demonstrating the sequence regions identified and the striking similarities of these regions across chromosomal locations is available. A custom microarray chip for these ICRs is also under development, to enable largescale screening, for example, to estimate the proportion of diseases with early origins or determine in adults whether stable marks can be used to augment screening algorithms. Such a custom chip can also be used to identify patterns associated with epigenetic response to early exposures. While such exposures are often transient, they nonetheless leave a ‘record’ of responses. Depending on the accumulation of these changes, they, in combination with underlying genetic factors and other exposures, could contribute to disease. The abilities to screen for past exposure marks, and also predict future risk of complex diseases, based on specific ‘molecular patterns’ detectable in accessible tissues, should accelerate the discovery of genomic regions of importance to disease and exposure, for closer interrogation in human disease studies.

We have established the contours of a screening tool that could be developed to detect a propensity for AD early, which will be especially useful when pharmaceutical or other interventions becomes available for significantly decelerating progression, improving quality of life and reducing costs of care. Our finding that 8% of AD-related DMRs and 5% of these are also similar in peripheral blood-derived DNA, implies that these ICRs can be found in DNA derived from peripheral tissues. Since these methylation marks are acquired before gastrulation and, despite drift with age, are stable over the life course, this implies that the custom methylation assay chip under development could be evaluated as a screening tool for AD, for triaging individuals for existing early pharmaceuticals that can stem progression. Although the number of differentially methylated regions associated with AD in AAs was ˜40 times higher than those found in EAs, and peripheral blood samples were not available from AA donors, the prospect of determining the feasibility of these marks for screening is being evaluated in an ongoing case-control study of AA and EA cases and controls.

These findings, however, should be interpreted in the context of their limitations. One limitation is that, in developing the algorithm putICR, used to identify methylation marks genome-wide, we used methylation fractions ranging from a liberal 30-70% (50±20%), to a more conservative 45%-55%, (50±5%). This approach precludes evaluation of a continuous pattern of methylation changes to define ICR boundaries. Since DNA methylation sequence data are available online, strategies such as change-point modeling have been used in copy number variant analyses and could be deployed, should a need for improved precision in ICR region boundaries arise. Another limitation is that cloned allele analysis, which would definitively define bona fide ICRs would need to be performed on all novel putative ICRs. While high throughput methods for cloned-allele analysis exist, a higher priority is to screen for ICRs of importance to disease or exposure, before cloned allele analyses are conducted. Consequently, we expect that not all 1,495 putative ICRs are bona fide ICRs; however, based on the characteristics of known ICRs, we are confident that >90% of human ICRs are captured in our sequence data. Finally, the lack of peripheral blood for AA AD donors presents a limitation, and provides a poignant example of why ethnic minorities should participate in biomedical research studies, despite the documented difficulties.

Despite these limitations, we provide the first draft of the human ICRs—the human imprintome—and have empirically demonstrated that ˜8% of Alzheimer's disease associated DMRs in the frontal cortex are within ICRs. This implies that these sequence regions are also present in accessible peripheral blood DNA, paving the way for a novel early-screening tool for AD.

Summarily, we generated a comprehensive compendium of all human Imprint Control Regions using whole genome bisulfite sequencing (WGBS) analysis of brain (ectoderm), kidney (mesoderm) and liver (endoderm) from banked embryonic tissue from nine conceptuses (27 libraries total), and analyzed the data to identify novel ICRs. These ICRs were identified based on the widely accepted assumptions that ICR methylation patterns are consistent across different tissues because they are established pre-gastrulation, ICR methylation patterns, once established, are stable over the life course. We also sequenced bisulfite-converted human sperm DNA, and downloaded WGBS data for human eggs from the Japanese Genotype-phenotype Archive (accession number JGAS00000000006). Data were analyzed in-house using a custom bioinformatic pipeline, putICR, to assess methylation and identify regions of the genome with approximately 50% (±15%) methylation, which would indicate parent of origin methylation. Using these criteria, we identified 1611 candidate ICRs, including 20 of the 24 previously characterized ICRs.

### REFERENCES

All references cited in the instant disclosure, including but not limited to all patents, patent applications and publications thereof, scientific journal articles, and database entries, are incorporated herein by reference in their entireties to the extent that they supplement, explain, provide a background for, or teach methodology, techniques, and/or compositions employed herein.
Altschul et al. (1990) Basic local alignment search tool. J Mol Biol 215: 403-410.
Ausubel et al. (2002) Short Protocols in Molecular Biology, Fifth ed. Wiley, New York, N.Y., United States of America.
Ausubel et al. (2003) Current Protocols in Molecular Biology, John Wylie & Sons, Inc., New York, N.Y., United States of America.
Batzer et al. (1991) Enhanced evolutionary PCR using oligonucleotides with inosine at the 3′-terminus. Nuc Acids Res 19:5081.
Bernal et al. (2013) Adaptive radiation-induced epigenetic alterations mitigated by antioxidants. FASEB J 27(2):665-71.
Cassidy & Charalambous (2018) Genomic imprinting, growth and maternal-fetal interactions. The Journal of Experimental Biology. 2018;221(Pt Suppl 1):jeb164517.

Crespi (2008) Genomic Imprinting in the Development and Evolution of Psychotic Spectrum Conditions. Biol Rev Camb Philos Soc 83(4):441-493.
Franks & McCarthy (2016) Exposing the exposures responsible for type 2 diabetes and obesity. Science. 354(6308):69-73.
Green et al. (2015) Expression of imprinted genes in placenta is associated with infant neurobehavioral development. Epigenetics 10(9):834-41.
Henikoff & Henikoff (1992) Amino Acid Substitution Matrices from Protein Blocks. Proc Natl Acad Sci U S A 89:10915-10919.
Hoyo et al. (2009) Imprint regulatory elements as epigenetic biosensors of exposure in epidemiological studies. J Epidemiol Community Health 63(9):683-684.
Ishida & Moore (2013) The role of imprinted genes in humans. Mol Aspects Med 34(4):826-840.
Jain et al. (2019) A combined miRNA-piRNA signature to detect Alzheimer's disease. Translational Psychiatry 9(1):250.
Jirtle & Skinner (2007) Environmental epigenomics and disease susceptibility. Nature Reviews 8(4):253-262.
Jirtle (1999) Genomic imprinting and cancer. Exp Cell Res 248(1):18-24.
Jirtle (2004) IGF2 loss of imprinting: a potential heritable risk factor for colorectal cancer. Gastroenterology 126(4):1190-1193.
Karlin & Altschul (1993) Applications and statistics for multiple high-scoring segments in molecular sequences. Proc Natl Acad Sci U S A 90(12):5873-5877.
Kitsiou-Tzeli & Tzetis (2017) Maternal epigenetics and fetal and neonatal growth. Curr Opin Endocrinol Diabetes Obes 24(1):43-46.
Krämer et al. (2019) How to copy and paste DNA microarrays. Sci Rep 9(1):13940.
Lambertini et al. (2012) Imprinted gene expression in fetal growth and development. Placenta 33(6):480-486.
Li et al. (2020) Potential role of genomic imprinted genes and brain developmental related genes in autism. BMC Medical Genomics 13:54.
Lorgen-Ritchie et al. (2019) Imprinting methylation in SNRPN and MEST1 in adult blood predicts cognitive ability. PLoS One 14(2):e0211799.
Luedi et al. (2007) Computational and experimental identification of novel human imprinted genes. Genome Res. 17(12):1723-1730.
Moran et al. (2016) Validation of a DNA methylation microarray for 850,000 CpG sites of the human genome enriched in enhancer sequences. Epigenomics 8(3):389-399.
Murphy et al. (2012) Differentially methylated regions of imprinted genes in prenatal, perinatal, and postnatal human tissues. PLoS One 7(7):e40924.
Murphy (2012) Targeting the epigenome in ovarian cancer. Future Oncol. 8(2):151-164.
Murrell et al. (2006) Association of apolipoprotein E genotype and Alzheimer disease in African Americans. Archives of Neurology 63(3):431-434.
Needleman & Wunsch (1970) A general method applicable to the search for similarities in the amino acid sequence of two proteins. J Mol Biol 48:443-453.
Ohtsuka et al. (1985) An alternative approach to deoxyoligonucleotides as hybridization probes by insertion of deoxyinosine at ambiguous codon positions. J Biol Chem 260:2605-2608.
Okae et al. (2014) Genome-Wide Analysis of DNA Methylation Dynamics during Early Human Development. PLoS genetics 10:e1004868.
Pearson & Lipman (1988) Improved tools for biological sequence comparison. Proc Natl Acad Sci USA 85:2444-2448.
Pigeyre et al. (2016) How obesity relates to socio-economic status: identification of eating behavior mediators. International Journal of Obesity 40(11):1794-1801.
Pirrung (2002) How to make a DNA chip. Angew Chem Int Ed Eng! 41(8):1276-1289.
Poelmans et al. (2013) AKAPs integrate genetic findings for autism spectrum disorders. Translational Psychiatry 3:e270.
Rossolini et al. (1994) Use of deoxyinosine-containing primers vs degenerate primers for polymerase chain reaction based on ambiguous sequence information. Mol Cell Probes 8:91-98.
Sandoval et al. (2011) Validation of a DNA methylation microarray for 450,000 CpG sites in the human genome. Epigenetics 6(6):692-702.
Seifan et al. (2015) Early Life Epidemiology of Alzheimer's Disease—A Critical Review. Neuroepidemiology 45:237-254.
Skaar et al. (2012) The human imprintome: regulatory mechanisms, methods of ascertainment, and roles in disease susceptibility. ILAR J 53(3-4):341-358.
Smith & Waterman (1981) Identification of common molecular subsequences. Adv Appl Math 2:482.
Snowdon et al. (1996) Linguistic Ability in Early Life and Cognitive Function and Alzheimer's Disease in Late Life. Findings From the Nun Study. JAMA 274:528-532.
Sun et al. (2014) MOABS: model based analysis of bisulfite sequencing data. Genome Biology 15(2):R38.
U.S. Patent Application Publication Nos. 2010/0056397, 2010/0304997, 2011/0105357, 2015/0232921.
U.S. Pat. Nos. 6,355,431; 6,429,027; 6,936,461; 7,824,917; 8,697,359 9,395,360; 9,688,971; 9,828,640.
Waterland (2003) Do maternal methyl supplements in mice affect DNA methylation of offspring? J Nutr. 133(1):238.

It will be understood that various details of the presently disclosed subject matter can be changed without departing from the scope of the presently disclosed subject matter. Furthermore, the foregoing description is for the purpose of illustration only, and not for the purpose of limitation.

1-6. (canceled)

7. A method for detecting a presence of and/or a susceptibility to a medical condition associated with monoallelic gene expression in a subject, the method comprising:
(a) obtaining a biological sample from the subject, wherein the biological sample comprises one or more nucleic acid molecules that correspond to an Imprint Control Region (ICR) selected from the group consisting of ICRs 1-1611, the genomic regions associated with SEQ ID NOs: 1612-1816, or a subset thereof;
(b) analyzing the one or more nucleic acid molecules to determine the DNA methylation status of one or both alleles of at least one imprinted gene associated with at least one of ICRs 1-1611 and/or the genomic regions associated with SEQ ID NOs: 1612-1816; and
(c) comparing the DNA methylation status of the one or both alleles of the at least one imprinted gene associated with the at least one of ICRs 1-1611 and/or the genomic regions associated with SEQ ID NOs: 1612-1816 to a control DNA methylation status,
wherein the comparing detects a presence of and/or a susceptibility to a medical condition associated with monoallelic gene expression in a subject.

8. The method of claim 7, wherein the DNA methylation status comprises one or more epigenomic features of at least one imprinted gene.

9. The method of claim 8, wherein the one or more epigenomic features comprises a methylation profile of the subject with respect to at least one imprinted gene.

10. The method of claim 9, wherein the one or more epigenomic features are selected from the group consisting of a DNA sequence methylation state, a nucleosome positioning feature, and a histone modification.

11. The method of claim 8, wherein the one or more epigenomic features relates to a gene for which expression or lack of expression is associated with the medical condition.

12. The method of claim 11, wherein the medical condition is Alzheimer's disease.

13. The method of claim 12, wherein at least one imprinted gene is selected from the set of genes associated with Alzheimer's disease, based on proximity to epigenomic features, correlated expression in association to epigenomic features, or reported association to Alzheimer disease in combination with either of the first two criteria.

14. The method of claim 7, wherein the biological sample comprises genomic DNA isolated from a cell, tissue, or organ of the subject, optionally a cell, tissue, or organ that is not affected by the medical condition.

15. A method for predicting a susceptibility to future development of a medical condition associated with monoallelic expression in a subject prior to the onset of any symptoms of the medical condition in the subject, the method comprising:
(a) obtaining a biological sample from the subject, wherein the biological sample comprises one or more nucleic acid molecules that correspond to an Imprint Control Region (ICR) selected from the group consisting of ICRs 1-1611 and/or the genomic regions associated with SEQ ID NOs: 1612-1816, or an informative subset thereof;
(b) analyzing the one or more nucleic acid molecules to determine the DNA methylation status of one or both alleles of at least one imprinted gene associated with at least one of ICRs 1-1611 and/or the genomic regions associated with SEQ ID NOs: 1612-1816; and
(c) determining whether the DNA methylation status determined correlates with future development of the medical condition,
whereby a susceptibility to future development of the medical condition is predicted.

16. The method of claim 15, wherein the DNA methylation status comprises one or more epigenomic features of at least one imprinted gene.

17. The method of claim 16, wherein one or more epigenomic features comprises a methylation profile of the subject with respect to at least one imprinted gene.

18. The method of claim 17, wherein one or more epigenomic features are selected from the group consisting of a DNA sequence methylation state, a nucleosome positioning feature, and a histone modification.

19. The method of claim 16, wherein the one or more epigenomic features relates to a gene for which expression or lack of expression is associated with the medical condition.

20. The method of claim 19, wherein the medical condition is Alzheimer's disease, autism, schizophrenia, or a tumor or cancer, optionally hepatocellular carcinoma.

21. The method of claim 18, wherein the at least one imprinted gene is selected from those listed in Tables 2 and 3.

22. The method of claim 15, wherein the biological sample comprises genomic DNA isolated from any cell, tissue, or organ of the subject, including a cell, tissue, or organ that is generally unaffected in subjects who have the medical condition, and not necessarily usable for diagnosis of conditions affecting target tissues by means specific to those affected tissues, including, but not limited to, physical morphology, immunological assays, protein expression, or RNA expression.

23. A nucleic acid array comprising one or more interrogatable nucleotide molecules, wherein the interrogatable nucleotide molecules are designed to allow identification of the DNA methylation status of ICRs that regulate one or more genes subject to monoalleleic expression in a biological sample isolated from a subject.

24. The nucleic acid array of claim 23, wherein nucleic acid array comprises, consists essentially of, or consists of a plurality of interrogatable nucleotide molecules that correspond to one or more of ICRs 1-1611 and/or the genomic regions associated with SEQ ID NOs: 1612-1816, optionally wherein the interrogatable nucleotide molecules comprise, consist essentially of, or consist of:
(a) target probes that comprise, consist essentially of, or consist of nucleotide sequences that are complementary to the nucleic acid sequences of ICRs 1-1611 and/or the genomic regions associated with SEQ ID NOs: 1612-1816 and/or an informative subset thereof; and
(b) target probes that comprise, consist essentially of, or consist of nucleotide sequences that are complementary to the nucleic acid sequences of ICRs 1-1611 and/or the genomic regions associated with SEQ ID NOs: 1612-1816 and/or the informative subset thereof subsequent to exposing the nucleic acid sequences of ICRs 1-1611 and/or the genomic regions associated with SEQ ID NOs: 1612-1816 and/or the informative subset thereof to a bisulfite converting treatment.

25. The nucleic acid array of claim 23, wherein the interrogatable nucleotide molecules can be interrogated with human genomic DNA.

26. The nucleic acid array of claim 23, wherein the plurality of interrogatable nucleotide molecules correspond to at least 100, 250, 500, 1000, or all of ICRs 1-1611 and/or the genomic regions associated with SEQ ID NOs: 1612-1816.
